(19) United States
(12) Patent Application Publication
(10) Pub. No.: US 2023/0133445 A1
(43) Pub. Date: May 4, 2023

(54) COMPOSITIONS AND METHODS FOR IMPROVING THE APPEARANCE OF SKIN

(71) Applicant: L'Oreal, Paris (FR)

(72) Inventors: I-Chien LIAO, Princeton, NJ (US); Rebecca BARRESI, Robbinsville, NJ (US); Charbel BOUEZ, Hoboken, NJ (US); Qian ZHENG, Bridgewater, NJ (US); Patricia BRIEVA, Manalapan, NJ (US); Stephen Matthew LYNCH, Westfield, NJ (US)

(21) Appl. No.: 17/976,631

(22) Filed: Oct. 28, 2022

Related U.S. Application Data
(60) Provisional application No. 63/273,743, filed on Oct. 29, 2021.
(60) Provisional application No. 63/293,045, filed on Dec. 22, 2021.

(30) Foreign Application Priority Data
Feb. 28, 2022 (FR) 2201765

(51) Int. Cl. A61K 8/42; A61K 8/365

(52) U.S. Cl. CPC A61K 8/42; A61K 8/365; A61K 2800/28; A61K 2800/88

(57) ABSTRACT

The disclosure relates to compositions, methods, regimens, and kits for improving the appearance of the skin by applying acetyl trifluoromethylphenyl valylglycine and at least one peeling agent to the skin.

### TECHNICAL FIELD

The disclosure relates to compositions for treating skin, methods for treating skin with skin treatment compositions, and kits containing skin treatment compositions.

### BACKGROUND

Aging can result in the appearance of wrinkles and other changes to the skin. The aging appearance of skin is caused, in part, by the disorganization of elastin and collagen fibers, which leads to a loss of elasticity, flexibility, and firmness.

Desquamation is a natural phenomenon that slows or reduces the effects of aging on the skin. It is related to the fact that the epidermis, which constitutes the upper layer of the skin, is in constant regeneration. The epidermis is made up of several cell layers, of which the deepest is the basal layer made up of undifferentiated cells. Over time, these cells will differentiate and migrate to the surface of the epidermis constituting the different layers of the epidermis, until the corneocytes (dead cells) form an outermost layer, the corneal layer (also called the stratum corneum), or the surface of the epidermis, which are dead cells that are removed by desquamation. This loss in surface is compensated by the migration of cells from the basal layer towards the surface of the epidermis resulting in perpetual skin renewal.

Forced removal of the corneal layer can accelerate desquamation, accelerate the renewal of the skin's surface, slow the effects of aging of skin, and/or improve the appearance of skin. It is known that skin peeling agents can remove the corneal layer. The term “peeling” conventionally denotes a controlled action of irritation of the surface of the skin with a chemical substance or via a physical process. A surface peeling treatment targets the epidermis and is based on a surface attack of the skin. It affects the epidermis either partially or entirely. A peeling treatment may consist, for example, in applying to the skin a chemical substance (a “chemical peeling agent”) for the purpose of bringing about limited and controlled destruction of the epidermis (and in some embodiments of the upper layers of the dermis). The treatment may require multiple applications, and the whole procedure may take anywhere from one or a few days to several weeks or months.

There are many different types of skin peel compositions, in terms of both concentration and specific peeling agent (i.e. active ingredient) used. For example, α-hydroxy acids such as lactic acid, glycolic acid, or the like, and β-hydroxy acids such as salicylic acid and its derivatives, are associated with the activation of desquamation. These agents promote exfoliation of the dead surface cells, which takes place less efficiently with age, in order to restore the qualities of the skin surface. At-home peels generally have lower concentrations of active ingredients (e.g. less than 15 wt % acids), and therefore primarily affect the most superficial layers of the skin. These peels are suitable for treating superficial wrinkles, general skin maintenance or non-severe skin issues. In contrast, professionally administered peels are typically more powerful, with greater concentrations of active ingredients (e.g. greater than 15 wt % acids). This enables these peels to target deeper levels of the dermis and treat moderate to severe wrinkling and other signs of aging.

There is an ongoing need for improved skin treatments that can increase desquamation and skin renewal. It has now been surprisingly and unexpectedly discovered that acetyl trifluoromethylphenyl valylglycine works synergistically with chemical peeling agents to increase desquamation and improve skin renewal, and thereby improve the appearance of the skin.

### SUMMARY

The instant disclosure relates to compositions and methods for treating skin, for example for stimulating, increasing, and/or improving skin renewal. For example, the disclosed treatments may work synergistically to encourage or improve desquamation, while helping reform a mature barrier through keratinization and upper terminal differentiation. According to certain aspects of the disclosure, methods and skin treatment compositions may advantageously enhance skin elasticity and/or reduce the appearance of wrinkles.

In various embodiments, the disclosure relates to methods for treating skin, for example skin on the face, neck, chest, and/or hands, the methods comprising applying to the skin (a) acetyl trifluoromethylphenyl valylglycine; and (b) at least one peeling agent, which is not limited, and can be chosen from, for example, dermabrasion, microdermabrasion, physical exfoliators, and/or chemical agents.

In various embodiments, the methods comprise applying acetyl trifluoromethylphenyl valylglycine in an amount ranging from about 0.01 to about 25 wt %, preferably from about 0.1 to about 20 wt %, more preferably from about 0.25 to about 15 wt %, most preferably from about 0.5 to about 10 wt %, to the skin, all amounts relative to the total weight of the composition in which the acetyl trifluoromethylphenyl valylglycine is present.

In various embodiments, the at least one chemical peeling agent may be chosen from α-hydroxy acids, β-hydroxy acids, trichloroacetic acid, or mixtures thereof. For example, in certain embodiments, the methods comprise applying at least one α-hydroxy acid to the skin, present in an amount ranging from about 0.1 to about 20 wt %, preferably from about 0.25 to about 15 wt %, more preferably from about 0.5 to about 10 wt %, most preferably from about 1 to about 5 wt %, or from about 2 to about 4 wt %, from about 4 to about 7 wt %, or from about 13 to about 16 wt %, relative to the total weight of the composition in which it is present. In some embodiments, the methods comprise applying to the skin a composition comprising from about 0.1 to about 25 wt %, preferably from about 1 to about 20 wt %, more preferably from about 2 to about 16 wt %, most preferably from about 3 to about 15 wt %, or about 2 to about 4 wt %, from about 4 to about 7 wt %, or from about 13 to about 16 wt %, glycolic acid, lactic acid, and/or derivatives thereof.

In other embodiments, the methods comprise applying to the skin a composition comprising at least one β-hydroxy acid, for example present in an amount ranging from about 0.01 to about 2 wt %, preferably from about 0.05 to about 2 wt %, more preferably from about 0.05 to about 1 wt %, most preferably from about 0.05 to about 0.5 wt %, relative to the total weight of the composition in which it is present. In further embodiments, the methods comprise applying to the skin a composition comprising salicylic acid or a derivative thereof, for example in an amount ranging from about 0.01 to about 2 wt %, preferably from about 0.05 to about 2 wt %, more preferably from about 0.05 to about 1 wt %, most preferably from about 0.05 to about 0.5 wt %, relative to the total weight of the composition in which it is present. In still further embodiments, the methods comprise applying to the skin a composition comprising trichloroacetic acid, for example in an amount ranging from about 1 to about 25 wt %, about 5 to about 22 wt %, about 7 to about 20 wt %, about 10 to about 18 wt %, or about 12 to about 16 wt %, relative to the total weight of the composition in which it is present.

Exemplary methods according to the disclosure comprise applying (a) acetyl trifluoromethylphenyl valylglycine to the skin before and/or after (b) at least one chemical peeling agent, such as, for example, within about 48 hours, within about 24 hours, within about 18 hours, within about 12 hours, within about 6 hours, within about 3 hours within about 2 hours, within about 1 hour, within about 45 minutes, within about 30 minutes, within about 20 minutes, or within about 10 minutes of one another. Multiple applications of either or both of the (a) acetyl trifluoromethylphenyl valylglycine and (b) at least one chemical peeling agent are contemplated. Further methods according to the disclosure comprise applying (a) acetyl trifluoromethylphenyl valylglycine to the skin before and/or treating the skin with a physical peeling agent, such as, for example, within about 48 hours, within about 24 hours, within about 18 hours, within about 12 hours, within about 6 hours, within about 3 hours within about 2 hours, within about 1 hour, within about 45 minutes, within about 30 minutes, within about 20 minutes, or within about 10 minutes of one another.

Certain methods according to the disclosure are methods of stimulating skin renewal, wherein the (a) acetyl trifluoromethylphenyl valylglycine, and the (b) at least one chemical peeling agent are applied to the skin in amounts effective to stimulate skin renewal. Certain methods according to the disclosure are methods of stimulating skin peeling, wherein the (a) acetyl trifluoromethylphenyl valylglycine, and the (b) at least one chemical peeling agent are applied to the skin in amounts effective to stimulate skin peeling. Certain methods according to the disclosure are methods of improving skin elasticity, wherein the (a) acetyl trifluoromethylphenyl valylglycine, and the (b) at least one chemical peeling agent are applied to the skin in amounts effective to improve skin elasticity. Certain methods according to the disclosure are methods of reducing elastase activity in skin, wherein the (a) acetyl trifluoromethylphenyl valylglycine, and the (b) at least one chemical peeling agent are applied to the skin in amounts effective to reduce elastase activity in skin. Certain methods according to the disclosure are methods of treating skin, wherein the (a) acetyl trifluoromethylphenyl valylglycine, and the (b) at least one chemical peeling agent are applied to the skin in amounts effective to improve the appearance of the skin.

Compositions comprising (a) acetyl trifluoromethylphenyl valylglycine, and (b) at least one chemical peeling agent are also disclosed. In some embodiments, compositions comprise (a) acetyl trifluoromethylphenyl valylglycine, and (b) at least one chemical peeling agent in amounts effective to improve the appearance of the skin, amounts effective to stimulate skin renewal of the skin, amounts effective to stimulate skin peeling, and/or amounts effective to improve skin elasticity.

Kits comprising (a) acetyl trifluoromethylphenyl valylglycine, and (b) at least one chemical peeling agent and/or implementation for exfoliation of skin are also disclosed.

### BRIEF DESCRIPTION OF THE FIGURES

Implementation of the present technology will now be described, by way of example only, with reference to the attached figures, wherein:

FIG. 1 shows synergistic inhibition of elastase activity by glycolic acid and/or acetyl trifluoromethylphenyl valylglycine.

FIG. 2 shows synergistic inhibition of elastase activity by lactic acid and/or acetyl trifluoromethylphenyl valylglycine.

FIG. 3 shows two-dimensional ex-vivo histological cross-sections of a) a skin sample treated with vehicle control (Control—2 wt % ethanol), b) a skin sample treated with 3 wt % lactic acid and 0.2 wt % salicylic acid (Peel A), c) a skin sample treated with 3 wt % glycolic acid and 2.4 wt % lactic acid (Peel B), d) a skin sample treated with a commercially-available formulation having 14.7 wt % glycolic acid and 0.1 wt % salicylic acid (Peel C), e) a skin sample treated with a commercially-available formulation having 15 wt % trichloroacetic acid and 3 wt % glycolic acid (Peel D), f) a skin sample treated with 3 wt % lactic acid and 0.2 wt % salicylic acid (Peel A), and 0.1 wt % acetyl trifluoromethylphenyl valylglycine, g) a skin sample treated with 3 wt % glycolic acid and 2.4 wt % lactic acid (Peel B), and 0.1 wt % acetyl trifluoromethylphenyl valylglycine, h) a skin sample treated with 14.7 wt % glycolic acid and 0.1 wt % salicylic acid (Peel C), and 0.1 wt % acetyl trifluoromethylphenyl valylglycine, and i) a skin sample treated with 15 wt % trichloroacetic acid and 3 wt % glycolic acid (Peel D), and 0.1 wt % acetyl trifluoromethylphenyl valylglycine. The cross-sections are stained for hematoxylin and eosin (H&E) expression.

FIG. 4 shows two-dimensional ex-vivo histological cross-sections of a) a skin sample treated with vehicle control (2 wt % ethanol), b) a skin sample treated with 3 wt % lactic acid and 0.2 wt % salicylic acid (Peel A), c) a skin sample treated with 3 wt % glycolic acid and 2.4 wt % lactic acid (Peel B), d) a skin sample treated with 14.7 wt % glycolic acid and 0.1 wt % salicylic acid (Peel C), e) a skin sample treated with 15 wt % trichloroacetic acid and 3 wt % glycolic acid (Peel D), f) a skin sample treated with 3 wt % lactic acid and 0.2 wt % salicylic acid (Peel A), and 0.1 wt % acetyl trifluoromethylphenyl valylglycine, g) a skin sample treated with 3 wt % glycolic acid and 2.4 wt % lactic acid (Peel B), and 0.1 wt % acetyl trifluoromethylphenyl valylglycine, h) a skin sample treated with 14.7 wt % glycolic acid and 0.1 wt % salicylic acid (Peel C), and 0.1 wt % acetyl trifluoromethylphenyl valylglycine, and i) a skin sample treated with 15 wt % trichloroacetic acid and 3 wt % glycolic acid (Peel D), and 0.1 wt % acetyl trifluoromethylphenyl valylglycine. The cross-sections are stained for Filaggrin (FLG) expression.

FIG. 5 shows two-dimensional ex-vivo histological cross-sections of a) a skin sample treated with vehicle control (2 wt % ethanol), b) a skin sample treated with 3 wt % lactic acid and 0.2 wt % salicylic acid (Peel A), c) a skin sample treated with 3 wt % glycolic acid and 2.4 wt % lactic acid (Peel B), d) a skin sample treated with 14.7 wt % glycolic acid and 0.1 wt % salicylic acid (Peel C), e) a skin sample treated with 15 wt % trichloroacetic acid and 3 wt % glycolic acid (Peel D), f) a skin sample treated with 3 wt % lactic acid and 0.2 wt % salicylic acid (Peel A), and 0.1 wt % acetyl trifluoromethylphenyl valylglycine, g) a skin sample treated with 3 wt % glycolic acid and 2.4 wt % lactic acid (Peel B), and 0.1 wt % acetyl trifluoromethylphenyl valylglycine, h) a skin sample treated with 14.7 wt % glycolic acid and 0.1 wt % salicylic acid (Peel C), and 0.1 wt % acetyl trifluoromethylphenyl valylglycine, and i) a skin sample treated with 15 wt % trichloroacetic acid and 3 wt % glycolic acid (Peel D), and 0.1 wt % acetyl trifluoromethylphenyl valylglycine. The cross-sections are stained for terminal deoxynucleotidyl transferase dUTP nick end labeling (TUNEL).

FIG. 6 shows two-dimensional ex-vivo histological cross-sections of a) a skin sample treated with vehicle control (2 wt % ethanol), b) a skin sample treated with 0.1 wt % acetyl trifluoromethylphenyl valylglycine, c) a skin sample treated with 15 wt % trichloroacetic acid and 3 wt % glycolic acid (Peel D), and d) a skin sample treated with 0.1 wt % acetyl trifluoromethylphenyl valylglycine, and 15 wt % trichloroacetic acid and 3 wt % glycolic acid (Peel D). The cross-sections are stained for hematoxylin and eosin. Scale Bar=100 μm.

FIG. 7 shows two-dimensional ex-vivo histological cross-sections of a) a skin sample treated with vehicle control (2 wt % ethanol), b) a skin sample treated with 0.1 wt % acetyl trifluoromethylphenyl valylglycine, c) a skin sample treated with 15 wt % trichloroacetic acid and 3 wt % glycolic acid (Peel D), and d) a skin sample treated with 0.1 wt % acetyl trifluoromethylphenyl valylglycine, and 15 wt % trichloroacetic acid and 3 wt % glycolic acid (Peel D). The cross-sections are stained for Filaggrin expression. Arrows point to altered stratum corneums.

FIG. 8 shows two-dimensional ex-vivo histological cross-sections of a) a skin sample treated with vehicle control (2 wt % ethanol), b) a skin sample treated with 0.1 wt % acetyl trifluoromethylphenyl valylglycine, c) a skin sample treated with 15 wt % trichloroacetic acid and 3 wt % glycolic acid (Peel D), and d) a skin sample treated with 0.1 wt % acetyl trifluoromethylphenyl valylglycine, and 15 wt % trichloroacetic acid and 3 wt % glycolic acid (Peel D). The cross-sections are stained for Desmocollin-1 (DSC1) expression. Arrows point to altered stratum corneums.

FIG. 9 shows two-dimensional ex-vivo histological cross-sections of a) a skin sample treated with vehicle control (2 wt % ethanol), b) a skin sample treated with 0.1 wt % acetyl trifluoromethylphenyl valylglycine, c) a skin sample treated with 15 wt % trichloroacetic acid and 3 wt % glycolic acid (Peel D), and d) a skin sample treated with 0.1 wt % acetyl trifluoromethylphenyl valylglycine, and 15 wt % trichloroacetic acid and 3 wt % glycolic acid (Peel D). The cross-sections are stained for kallikrein-related peptidase 5 (KLK5) (also known as stratum corneum tryptic enzyme (SCTE)) expression. Arrows point to altered stratum corneums.

FIG. 10 shows two-dimensional ex-vivo histological cross-sections of a) a skin sample treated with vehicle control (2 wt % ethanol), b) a skin sample treated with 0.1 wt % acetyl trifluoromethylphenyl valylglycine, c) a skin sample treated with 15 wt % trichloroacetic acid and 3 wt % glycolic acid (Peel D), and d) a skin sample treated with 0.1 wt % acetyl trifluoromethylphenyl valylglycine, and 15 wt % trichloroacetic acid and 3 wt % glycolic acid (Peel D). The cross-sections are stained for terminal deoxynucleotidyl transferase dUTP nick end labeling expression.

FIG. 11 shows the amount of a 2% solution of acetyl trifluoromethylphenyl valylglycine in a) the stratum corneum (SC), b) the epidermis (EP), c) the dermis (D), d) the receptor fluid (RF), and e) the amount that passes through the stratum corneum into the skin (EP+D+RF), after application to skin samples using a Franz cell horizontal cell clamp system.

FIG. 12 shows (A) a home-care product regimen for 2 study groups (Groups 1-2), and (B) treatment phases for Groups 1 and 2 of Example 4.

FIG. 13 shows a chart comparing the clinical outcomes of a 2 week, 4 week, 6 week, 10 week, and 12 week regimen described in Example 4 with Group 1 undergoing treatment with 0.3% retinol and Group 2 undergoing treatment with 2% solution of acetyl trifluoromethylphenyl valylglycine and a 0.45% salicylic acid/11.11% lactic acid at home chemical peel. Checkmarks (“√”) denote a statistically significant improvement in outcomes for Group 2 over Group 1 or Group 1 over Group 2. Equals signs (“=”) denote a statistically insignificant difference in outcomes between Group 1 and Group 2.

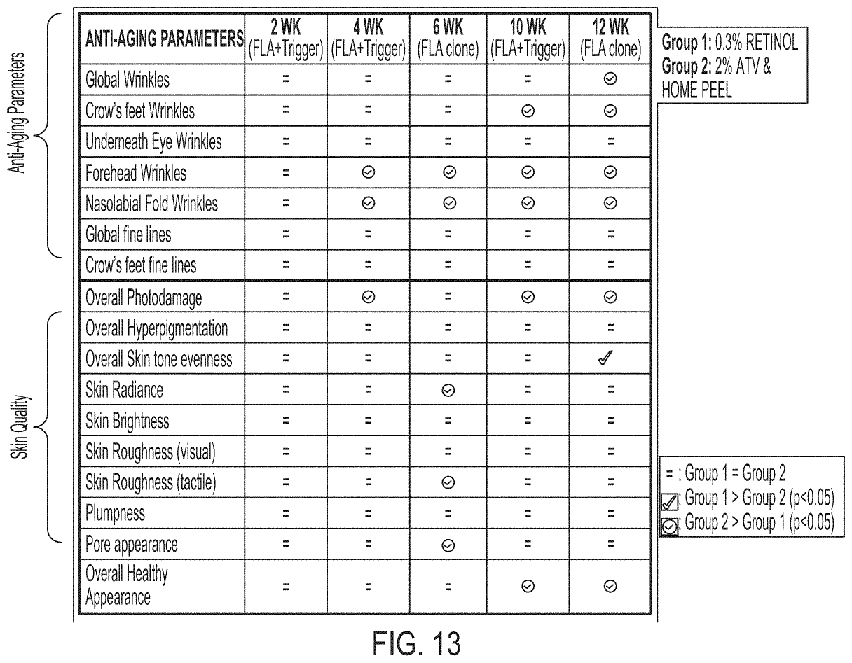

FIG. 14 shows a chart comparing the clinical outcomes of a 2 week, 6 week, and 12 week regimen described in Example 4 with Group 1 undergoing treatment with 0.3% retinol and Group 2 undergoing treatment with 2% solution of acetyl trifluoromethylphenyl valylglycine and a 0.45% salicylic acid/11.11% lactic acid chemical peel as compared to baseline. Blank boxes denote statistically insignificant differences in clinical outcomes of Group 1 or Group 2 over baseline. Boxes with dots denote statistically significant (p<0.05) improvements in clinical outcomes for Group 1 or Group 2 over baseline. Boxes with solid diagonal lines denote statistically significant and clinically significant (p<0.05) improvements in outcomes for Group 1 or Group 2 over baseline.

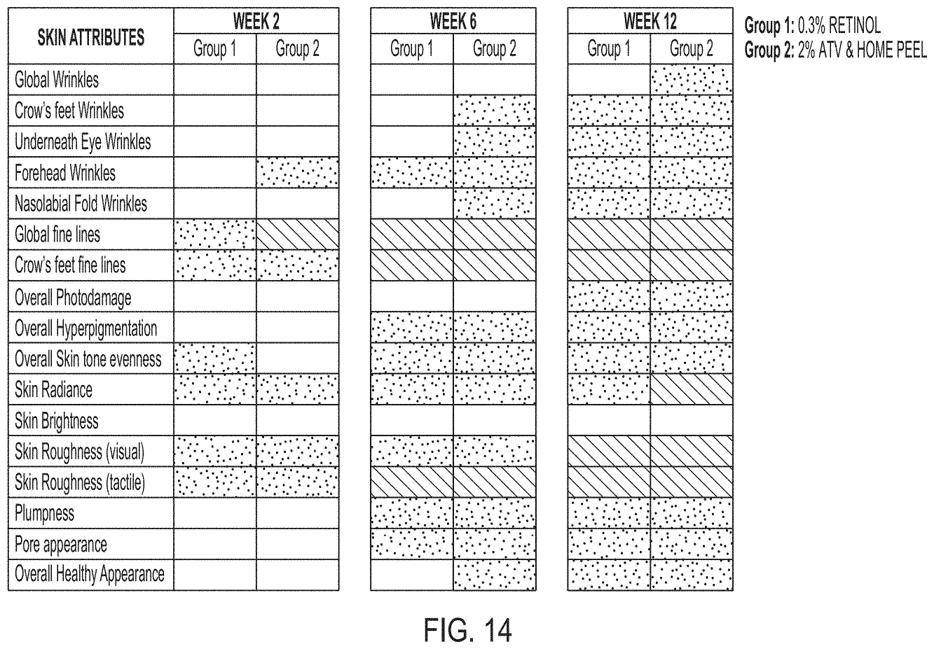

FIG. 15 shows a graph of clinical grading for global fine lines between treatment Groups 1 and 2 over the 12-week regimen of Example 4.

FIG. 16 shows a graph of clinical grading for nasolabial folds between treatment Groups 1 and 2 over the 12-week regimen of Example 4. An asterisk (“*”) denotes a statistically significant different (p<0.05) in nasolabial fold wrinkles compared to Group 1 at the respective timepoint.

FIG. 17 shows a graph of clinical grading for photodamage between treatment Groups 1 and 2 over the 12-week regimen of Example 4. An asterisk (“*”) denotes a statistically significant different (p<0.05) in photodamage compared to Group 1 at the respective timepoint.

FIG. 18 shows a graph of clinical grading for under eye wrinkles between treatment Groups 1 and 2 over the 12-week regimen of Example 4.

FIG. 19 shows a graph of clinical grading for overall healthy appearance between treatment Groups 1 and 2 over the 12-week regimen of Example 4. An asterisk (“*”) denotes a statistically significant different (p<0.05) in overall healthy appearance compared to Group 1 at the respective timepoint.

It should be understood that the various aspects are not limited to the arrangements and instrumentality shown in the drawings.

### DETAILED DESCRIPTION

The disclosure relates to compositions, methods, and kits for treating skin, such as by increasing, accelerating, and/or improving skin renewal, and/or improving the appearance of skin. The compositions according to various aspects of the disclosure comprise acetyl trifluoromethylphenyl valylglycine and/or at least one chemical peeling agent. The methods according to various aspects of the disclosure comprise applying to skin, simultaneously or sequentially, acetyl trifluoromethylphenyl valylglycine and at least one chemical peeling agent. Kits according to various aspects of the disclosure comprise acetyl trifluoromethylphenyl valylglycine and at least one chemical peeling agent.

The compositions and methods according to various aspects of the disclosure may protect against or slow degradation of skin elastase, improve skin elasticity, decrease sagging, improve or accelerate skin turnover/desquamation of the skin, stimulate or improve epidermal renewal, increase exfoliation, reduce or slow the appearance of skin aging (such as wrinkles), increase skin keratinization or upper terminal differentiation, and/or may smooth skin. For example, the skin treatment methods generally include applying acetyl trifluoromethylphenyl valylglycine and at least one chemical peeling agent to skin to accelerate desquamation and cell turnover in the epidermis layer. Surprisingly, it has been found that in some embodiments, the skin treatment methods and compositions according to the disclosure inhibit elastase activity.

In various embodiments, acetyl trifluoromethylphenyl valylglycine and the at least one chemical peeling agent work synergistically to improve the appearance of the skin. By way of example, the skin treatment methods and compositions disclosed herein may be effective in reducing the appearance of wrinkles by the acceleration of cell turnover, for example, by keratinization and upper terminal differentiation. By way of further example, the skin treatment methods and compositions disclosed herein may be effective in reducing the appearance of wrinkles by the inhibition of elastase, thereby allowing for the growth of elastin fibers as opposed to their breakdown by these enzymes, thus improving skin elasticity.

A more detailed description of exemplary compositions, methods, and kits for treating skin according to the disclosure is provided below.

### I. Compositions

Compositions according to various embodiments of the disclosure comprise acetyl trifluoromethylphenyl valylglycine and/or at least one peeling agent, which may in some embodiments be chosen from chemical peeling agents. In certain embodiments, compositions according to the disclosure comprise both acetyl trifluoromethylphenyl valylglycine and at least one chemical peeling agent. In other embodiments, compositions according to the disclosure comprise acetyl trifluoromethylphenyl valylglycine or at least one chemical peeling agent.

Acetyl Trifluoromethylphenyl Valylglycine

According to various embodiments, compositions according to the disclosure comprise at least about 0.01 wt % of acetyl trifluoromethylphenyl valylglycine, such as from about 0.01 to about 25 wt %, relative to the total weight of the composition in which it is present. For example, the amount of acetyl trifluoromethylphenyl valylglycine may range from about 0.1 to about 25 wt %, about 0.1 to about 22 wt %, about 0.1 to about 19 wt %, about 0.1 to about 16 wt %, about 0.1 to about 14 wt %, about 0.1 to about 12 wt %, about 0.1 to about 10 wt %, about 0.1 to about 8 wt %, about 0.1 to about 6 wt %, about 0.1 to about 5 wt %, about 0.1 to about 4 wt %, about 0.1 to about 3 wt %, about 0.1 to about 2 wt %, about 0.1 to about 1 wt %; about 0.25 to about 25 wt %, about 0.25 to about 22 wt %, about 0.25 to about 19 wt %, about 0.25 to about 16 wt %, about 0.25 to about 14 wt %, about 0.25 to about 12 wt %, about 0.25 to about 10 wt %, about 0.25 to about 8 wt %, about 0.25 to about 6 wt %, about 0.25 to about 5 wt %, about 0.25 to about 4 wt %, about 0.25 to about 3 wt %, about 0.25 to about 2 wt %, about 0.25 to about 1 wt %; about 0.5 to about 25 wt %, about 0.5 to about 22 wt %, about 0.5 to about 19 wt %, about 0.5 to about 16 wt %, about 0.5 to about 14 wt %, about 0.5 to about 12 wt %, about 0.5 to about 10 wt %, about 0.5 to about 8 wt %, about 0.5 to about 6 wt %, about 0.5 to about 5 wt %, about 0.5 to about 4 wt %, about 0.5 to about 3 wt %, about 0.5 to about 2 wt %, about 0.5 to about 1 wt %; about 0.75 to about 25 wt %, about 0.75 to about 22 wt %, about 0.75 to about 19 wt %, about 0.75 to about 16 wt %, about 0.75 to about 14 wt %, about 0.75 to about 12 wt %, about 0.75 to about 10 wt %, about 0.75 to about 8 wt %, about 0.75 to about 6 wt %, about 0.75 to about 5 wt %, about 0.75 to about 4 wt %, about 0.75 to about 3 wt %, about 0.75 to about 2 wt %; about 1 to about 25 wt %, about 1 to about 22 wt %, about 1 to about 19 wt %, about 1 to about 16 wt %, about 1 to about 14 wt %, about 1 to about 12 wt %, about 1 to about 10 wt %, about 1 to about 8 wt %, about 1 to about 6 wt %, about 1 to about 5 wt %, about 1 to about 4 wt %, about 1 to about 3 wt %, about 1 to about 2 wt %; about 2 to about 25 wt %, about 2 to about 22 wt %, about 2 to about 19 wt %, about 2 to about 16 wt %, about 2 to about 14 wt %, about 2 to about 12 wt %, about 2 to about 10 wt %, about 2 to about 8 wt %, about 2 to about 6 wt %, about 2 to about 5 wt %, about 2 to about 4 wt %, about 2 to about 3 wt %; about 5 to about 25 wt %, about 5 to about 22 wt %, about 5 to about 19 wt %, about 5 to about 16 wt %, about 5 to about 14 wt %, about 5 to about 12 wt %, about 5 to about 10 wt %, about 5 to about 8 wt %; about 7.5 to about 27.5 wt %, about 7.5 to about 22 wt %, about 7.5 to about 19 wt %, about 7.5 to about 16 wt %, about 7.5 to about 14 wt %, about 7.5 to about 12 wt %, about 7.5 to about 10 wt %; about 10 to about 27.5 wt %, about 10 to about 22 wt %, about 10 to about 19 wt %, about 10 to about 16 wt %, about 10 to about 14 wt %, about 10 to about 12 wt %; about 15 to about 27.5 wt %, about 15 to about 22 wt %, about 15 to about 19 wt %; about 20 to about 27.5 wt %, or about 20 to about 22 wt %, including ranges and subranges therebetween, relative to the total weight of the composition in which it is present.

In some embodiments, the composition comprises an amount of acetyl trifluoromethylphenyl valylglycine such that about 0.001 to about 2.5 wt % of acetyl trifluoromethylphenyl valylglycine penetrates through the epidermis and reaches the dermal layer of the skin. For example, the composition may comprise an amount of acetyl trifluoromethylphenyl valylglycine such that about 0.002 to about 2 wt %, about 0.002 to about 1.9 wt %, about 0.002 to about 1.8 wt %, about 0.002 to about 1.7 wt %, about 0.002 to about 1.6 wt %, about 0.002 to about 1.5 wt %, about 0.002 to about 1.4 wt %, about 0.002 to about 1.3 wt %, about 0.002 to about 1.2 wt %, about 0.002 to about 1.1 wt %, about 0.002 to about 1 wt %, about 0.002 to about 0.9 wt %, about 0.002 to about 0.8 wt %, about 0.002 to about 0.7 wt %, about 0.002 to about 0.6 wt %, about 0.002 to about 0.5 wt %, about 0.002 to about 0.4 wt %, about 0.002 to about 0.3 wt %, about 0.002 to about 0.2 wt %, about 0.002 to about 0.1 wt %, about 0.002 to about 0.09 wt %, about 0.002 to about 0.08 wt %, about 0.002 to about 0.07 wt %, about 0.002 to about 0.06 wt %, about 0.002 to about 0.05 wt %, about 0.0042 to about 0.04 wt %, about 0.002 to about 0.03 wt %, about 0.002 to about 0.02 wt %, about 0.002 to about 0.01 wt %, about 0.01 to about 2 wt %, about 0.01 to about 1.9 wt %, about 0.01 to about 1.8 wt %, about 0.01 to about 1.7 wt %, about 0.01 to about 1.6 wt %, about 0.01 to about 1.5 wt %, about 0.01 to about 1.4 wt %, about 0.01 to about 1.3 wt %, about 0.01 to about 1.2 wt %, about 0.01 to about 1.1 wt %, about 0.01 to about 1 wt %, about 0.01 to about 0.9 wt %, about 0.01 to about 0.8 wt %, about 0.01 to about 0.7 wt %, about 0.01 to about 0.6 wt %, about 0.01 to about 0.5 wt %, about 0.01 to about 0.4 wt %, about 0.01 to about 0.3 wt %, about 0.01 to about 0.2 wt %, about 0.01 to about 0.1 wt %, about 0.01 to about 0.09 wt %, about 0.01 to about 0.08 wt %, about 0.01 to about 0.07 wt %, about 0.01 to about 0.06 wt %, about 0.01 to about 0.05 wt %, about 0.01 to about 0.04 wt %, about 0.01 to about 0.03 wt %, about 0.01 to about 0.02 wt %, about 0.01 to about 0.01 wt %, about 0.006 to about 2 wt %, about 0.006 to about 1.9 wt %, about 0.006 to about 1.8 wt %, about 0.006 to about 1.7 wt %, about 0.006 to about 1.6 wt %, about 0.006 to about 1.5 wt %, about 0.006 to about 1.4 wt %, about 0.006 to about 1.3 wt %, about 0.006 to about 1.2 wt %, about 0.006 to about 1.1 wt %, about 0.006 to about 1 wt %, about 0.006 to about 0.9 wt %, about 0.006 to about 0.8 wt %, about 0.006 to about 0.7 wt %, about 0.006 to about 0.6 wt %, about 0.006 to about 0.5 wt %, about 0.006 to about 0.4 wt %, about 0.006 to about 0.3 wt %, about 0.006 to about 0.2 wt %, about 0.006 to about 0.1 wt %, about 0.006 to about 0.09 wt %, about 0.006 to about 0.08 wt %, about 0.006 to about 0.07 wt %, about 0.006 to about 0.06 wt %, about 0.006 to about 0.05 wt %, about 0.006 to about 0.04 wt %, about 0.006 to about 0.03 wt %, about 0.006 to about 0.02 wt %, about 0.006 to about 0.01 wt %, about 0.008 to about 2 wt %, about 0.008 to about 1.9 wt %, about 0.008 to about 1.8 wt %, about 0.008 to about 1.7 wt %, about 0.008 to about 1.6 wt %, about 0.008 to about 1.5 wt %, about 0.008 to about 1.4 wt %, about 0.008 to about 1.3 wt %, about 0.008 to about 1.2 wt %, about 0.008 to about 1.1 wt %, about 0.008 to about 1 wt %, about 0.008 to about 0.9 wt %, about 0.008 to about 0.8 wt %, about 0.008 to about 0.7 wt %, about 0.008 to about 0.6 wt %, about 0.008 to about 0.5 wt %, about 0.008 to about 0.4 wt %, about 0.008 to about 0.3 wt %, about 0.008 to about 0.2 wt %, about 0.008 to about 0.1 wt %, about 0.008 to about 0.09 wt %, about 0.008 to about 0.08 wt %, about 0.008 to about 0.07 wt %, about 0.008 to about 0.06 wt %, about 0.008 to about 0.05 wt %, about 0.008 to about 0.04 wt %, about 0.008 to about 0.03 wt %, about 0.008 to about 0.02 wt %, about 0.008 to about 0.01 wt %, about 0.01 to about 2 wt %, about 0.01 to about 1.9 wt %, about 0.01 to about 1.8 wt %, about 0.01 to about 1.7 wt %, about 0.01 to about 1.6 wt %, about 0.01 to about 1.5 wt %, about 0.01 to about 1.4 wt %, about 0.01 to about 1.3 wt %, about 0.01 to about 1.2 wt %, about 0.01 to about 1.1 wt %, about 0.01 to about 1 wt %, about 0.01 to about 0.9 wt %, about 0.01 to about 0.8 wt %, about 0.01 to about 0.7 wt %, about 0.01 to about 0.6 wt %, about 0.01 to about 0.5 wt %, about 0.01 to about 0.4 wt %, about 0.01 to about 0.3 wt %, about 0.01 to about 0.2 wt %, about 0.01 to about 0.1 wt %, about 0.01 to about 0.09 wt %, about 0.01 to about 0.08 wt %, about 0.01 to about 0.07 wt %, about 0.01 to about 0.06 wt %, about 0.01 to about 0.05 wt %, about 0.01 to about 0.04 wt %, about 0.01 to about 0.03 wt %, about 0.01 to about 0.02 wt %, about 0.025 to about 2 wt %, about 0.025 to about 1.9 wt %, about 0.025 to about 1.8 wt %, about 0.025 to about 1.7 wt %, about 0.025 to about 1.6 wt %, about 0.025 to about 1.5 wt %, about 0.025 to about 1.4 wt %, about 0.025 to about 1.3 wt %, about 0.025 to about 1.2 wt %, about 0.025 to about 1.1 wt %, about 0.025 to about 1 wt %, about 0.025 to about 0.9 wt %, about 0.025 to about 0.8 wt %, about 0.025 to about 0.7 wt %, about 0.025 to about 0.6 wt %, about 0.025 to about 0.5 wt %, about 0.025 to about 0.4 wt %, about 0.025 to about 0.3 wt %, about 0.025 to about 0.2 wt %, about 0.025 to about 0.1 wt %, about 0.025 to about 0.09 wt %, about 0.025 to about 0.08 wt %, about 0.025 to about 0.07 wt %, about 0.025 to about 0.06 wt %, about 0.025 to about 0.05 wt %, about 0.025 to about 0.04 wt %, about 0.05 to about 2 wt %, about 0.05 to about 1.9 wt %, about 0.05 to about 1.8 wt %, about 0.05 to about 1.7 wt %, about 0.05 to about 1.6 wt %, about 0.05 to about 1.5 wt %, about 0.05 to about 1.4 wt %, about 0.05 to about 1.3 wt %, about 0.05 to about 1.2 wt %, about 0.05 to about 1.1 wt %, about 0.05 to about 1 wt %, about 0.05 to about 0.9 wt %, about 0.05 to about 0.8 wt %, about 0.05 to about 0.7 wt %, about 0.05 to about 0.6 wt %, about 0.05 to about 0.5 wt %, about 0.05 to about 0.4 wt %, about 0.05 to about 0.3 wt %, about 0.05 to about 0.2 wt %, about 0.05 to about 0.1 wt %, about 0.05 to about 0.09 wt %, about 0.05 to about 0.08 wt %, about 0.05 to about 0.07 wt %, about 0.06 to about 2 wt %, about 0.06 to about 1.9 wt %, about 0.06 to about 1.8 wt %, about 0.06 to about 1.7 wt %, about 0.06 to about 1.6 wt %, about 0.06 to about 1.5 wt %, about 0.06 to about 1.4 wt %, about 0.06 to about 1.3 wt %, about 0.06 to about 1.2 wt %, about 0.06 to about 1.1 wt %, about 0.06 to about 1 wt %, about 0.06 to about 0.9 wt %, about 0.06 to about 0.8 wt %, about 0.06 to about 0.7 wt %, about 0.06 to about 0.6 wt %, about 0.06 to about 0.5 wt %, about 0.06 to about 0.4 wt %, about 0.06 to about 0.3 wt %, about 0.06 to about 0.2 wt %, about 0.06 to about 0.1 wt %, about 0.06 to about 0.09 wt %, about 0.06 to about 0.08 wt %, about 0.06 to about 0.07 wt %, about 0.07 to about 2 wt %, about 0.07 to about 1.9 wt %, about 0.07 to about 1.8 wt %, about 0.07 to about 1.7 wt %, about 0.07 to about 1.6 wt %, about 0.07 to about 1.5 wt %, about 0.07 to about 1.4 wt %, about 0.07 to about 1.3 wt %, about 0.07 to about 1.2 wt %, about 0.07 to about 1.1 wt %, about 0.07 to about 1 wt %, about 0.07 to about 0.9 wt %, about 0.07 to about 0.8 wt %, about 0.07 to about 0.7 wt %, about 0.07 to about 0.6 wt %, about 0.07 to about 0.5 wt %, about 0.07 to about 0.4 wt %, about 0.07 to about 0.3 wt %, about 0.07 to about 0.2 wt %, about 0.07 to about 0.1 wt %, about 0.07 to about 0.09 wt %, about 0.07 to about 0.08 wt %, about 0.08 to about 2 wt %, about 0.08 to about 1.9 wt %, about 0.08 to about 1.8 wt %, about 0.08 to about 1.7 wt %, about 0.08 to about 1.6 wt %, about 0.08 to about 1.5 wt %, about 0.08 to about 1.4 wt %, about 0.08 to about 1.3 wt %, about 0.08 to about 1.2 wt %, about 0.08 to about 1.1 wt %, about 0.08 to about 1 wt %, about 0.08 to about 0.9 wt %, about 0.08 to about 0.8 wt %, about 0.08 to about 0.7 wt %, about 0.08 to about 0.6 wt %, about 0.08 to about 0.5 wt %, about 0.08 to about 0.4 wt %, about 0.08 to about 0.3 wt %, about 0.08 to about 0.2 wt %, about 0.08 to about 0.1 wt %, about 0.08 to about 0.09 wt %, about 0.09 to about 2 wt %, about 0.09 to about 1.9 wt %, about 0.09 to about 1.8 wt %, about 0.09 to about 1.7 wt %, about 0.09 to about 1.6 wt %, about 0.09 to about 1.5 wt %, about 0.09 to about 1.4 wt %, about 0.09 to about 1.3 wt %, about 0.09 to about 1.2 wt %, about 0.09 to about 1.1 wt %, about 0.09 to about 1 wt %, about 0.09 to about 0.9 wt %, about 0.09 to about 0.8 wt %, about 0.09 to about 0.7 wt %, about 0.09 to about 0.6 wt %, about 0.09 to about 0.5 wt %, about 0.09 to about 0.4 wt %, about 0.09 to about 0.3 wt %, about 0.09 to about 0.2 wt %, about 0.09 to about 0.1 wt %, about 0.1 to about 2 wt %, about 0.1 to about 1.9 wt %, about 0.1 to about 1.8 wt %, about 0.1 to about 1.7 wt %, about 0.1 to about 1.6 wt %, about 0.1 to about 1.5 wt %, about 0.1 to about 1.4 wt %, about 0.1 to about 1.3 wt %, about 0.1 to about 1.2 wt %, about 0.1 to about 1.1 wt %, about 0.1 to about 1 wt %, about 0.1 to about 0.9 wt %, about 0.1 to about 0.8 wt %, about 0.1 to about 0.7 wt %, about 0.1 to about 0.6 wt %, about 0.1 to about 0.5 wt %, about 0.1 to about 0.4 wt %, about 0.1 to about 0.3 wt %, or about 0.1 to about 0.2 wt %, including all ranges and subranges therebetween, of acetyl trifluoromethylphenyl valylglycine penetrates the epidermis and reaches the dermal layer of the skin. In some embodiments, the composition comprises an amount of acetyl trifluoromethylphenyl valylglycine such that about 0.01 to about 1.5 wt %, preferably about 0.025 to about 1.3 wt %, more preferably about 0.5 to about 1.1 wt %, most preferably about 0.6 to about 1 wt % of acetyl trifluoromethylphenyl valylglycine penetrates the epidermis and reaches the dermal layer of the skin.

In various embodiments, compositions according to the disclosure comprise acetyl trifluoromethylphenyl valylglycine but are free or substantially free of chemical peeling agents.

Peeling Agents

According to various embodiments, compositions according to the disclosure may comprise at least one peeling agent, which can be chosen from, for example, dermabrasion, microdermabrasion, physical exfoliators, and/or chemical peeling agents. Physical exfoliators may include, for example, microbeads or other small particles. In some embodiments, the peeling agents are physical and not comprised in a composition, for example exfoliating pads that may or may not comprise a chemical peeling agent, or other exfoliating devices.

Exemplary and non-limiting chemical peeling agents that may be used include chemical agents capable of acting either directly on peeling by encouraging exfoliation, such as β-hydroxyacids (BHAs), for example salicylic acid and derivatives thereof (including n-octanoyl 5-salicylic acid, otherwise known as capryloyl salicylic acid (INCI name)); α-hydroxyacids (AHAs), such as glycolic, citric, lactic, tartaric, malic, or mandelic acids; 8-hexadecene-1,16-dicarboxylic acid, or 9-octadecene dioic acid; urea and derivatives thereof; trichloroacetic acid; gentisic acid and derivatives thereof; oligofructoses; cinnamic acid; extract of Saphora japonica; resveratrol; certain derivatives of jasmonic acid; resorcinol; phenol; or mixtures thereof.

Further exemplary and non-limiting chemical peeling agents that may be used include compounds involved in peeling or degrading corneodesmosomes, such as aminosulfonic compounds, e.g. 4-(2-hydroxyethyl)piperazine-1-propanesulfonic acid (HEPES); 2-oxothiazolidine-4-carboxylic acid (procysteine) and derivatives thereof; the derivatives of α-amino acids of the glycine type (as described in EP-0 852 949) and also sodium methyl glycine diacetate sold by BASF under the trade name Trilon M; honey; derivatives of sugar such as O-octanoyl-6-D-maltose and N-acetyl glucosamine; or mixtures thereof.

As still further exemplary and non-limiting chemical peeling agents suitable for use in compositions according to the disclosure, mention be made of EDTA and derivatives thereof, Laminaria extracts, O-linoleyl-6-D-glucose; (3-hydroxy-2-pentylcyclopentyl) acetic acid, glycerol trilactate, S carboxymethyl cysteine, silica-containing derivatives of salicylate such as those described in patent EP 0 796 861, oligofructoses such as those described in patent EP 0 218 200, agents having effects on transglutaminase as in patent EP 0 899 330; extract of the flower ficus Opuntia indica such as Exfolactive® from Silab; 8-hexadecene 1,16-dicarboxylic acid; esters of glucose and of vitamin F; or mixtures thereof.

The chemical peeling agent(s) may, in certain embodiments, be chosen from α-hydroxy acids such as citric, lactic, glycolic, malic, tartaric, or mandelic acids; β-hydroxy acids such as salicylic acid or derivatives thereof, e.g. n-octanoyl-5-salicylic acid; trichloroacetic acid; or mixtures thereof.

The total amount of chemical peeling agent(s) present in the composition may range from about 0.01 to about 25 wt %, such as from about 0.1 to about 20 wt %, relative to the total weight of the composition in which it is present. For example, the total amount of chemical peeling agent present in the composition may range from about 0.5 to about 25 wt %, about 0.5 to about 22 wt %, about 0.5 to about 19 wt %, about 0.5 to about 16 wt %, about 0.5 to about 14 wt %, about 0.5 to about 12 wt %, about 0.5 to about 10 wt %, about 0.5 to about 8 wt %, about 0.5 to about 6 wt %, about 0.5 to about 5 wt %, about 0.5 to about 4 wt %, about 0.5 to about 3 wt %, about 0.5 to about 2 wt %, about 0.5 to about 1 wt %; about 1 to about 25 wt %, about 1 to about 22 wt %, about 1 to about 19 wt %, about 1 to about 16 wt %, about 1 to about 14 wt %, about 1 to about 12 wt %, about 1 to about 10 wt %, about 1 to about 8 wt %, about 1 to about 6 wt %, about 1 to about 5 wt %, about 1 to about 4 wt %, about 1 to about 3 wt %, about 1 to about 2 wt %; about 2 to about 25 wt %, about 2 to about 22 wt %, about 2 to about 19 wt %, about 2 to about 16 wt %, about 2 to about 14 wt %, about 2 to about 12 wt %, about 2 to about 10 wt %, about 2 to about 8 wt %, about 2 to about 6 wt %, about 2 to about 5 wt %, about 2 to about 4 wt %, about 2 to about 3 wt %; about 3 to about 25 wt %, about 3 to about 22 wt %, about 3 to about 19 wt %, about 3 to about 16 wt %, about 3 to about 14 wt %, about 3 to about 12 wt %, about 3 to about 10 wt %, about 3 to about 8 wt %, about 3 to about 6 wt %, about 3 to about 5 wt %, about 3 to about 4 wt %, about 4 to about 25 wt %, about 4 to about 22 wt %, about 4 to about 19 wt %, about 4 to about 16 wt %, about 4 to about 14 wt %, about 4 to about 12 wt %, about 4 to about 10 wt %, about 4 to about 8 wt %, about 4 to about 6 wt %, about 4 to about 5 wt %, about 5 to about 25 wt %, about 5 to about 22 wt %, about 5 to about 19 wt %, about 5 to about 16 wt %, about 5 to about 14 wt %, about 5 to about 12 wt %, about 5 to about 10 wt %, about 5 to about 8 wt %; about 7.5 to about 25 wt %, about 7.5 to about 22 wt %, about 7.5 to about 19 wt %, about 7.5 to about 16 wt %, about 7.5 to about 14 wt %, about 7.5 to about 12 wt %, about 7.5 to about 10 wt %; about 10 to about 25 wt %, about 10 to about 22 wt %, about 10 to about 19 wt %, about 10 to about 16 wt %, about 10 to about 14 wt %, about 10 to about 12 wt %; about 12 to about 25 wt %, about 12 to about 22 wt %, about 12 to about 19 wt %, about 12 to about 16 wt %, about 12 to about 14 wt %, about 15 to about 25 wt %, about 15 to about 22 wt %, about 15 to about 19 wt %, about 17 to about 25 wt %, about 17 to about 22 wt %, or about 17 to about 19 wt %, including ranges and subranges therebetween, relative to the total weight of the composition in which it is present.

For example, in various embodiments, compositions according to the disclosure comprise at least one AHA as a chemical peeling agent. In certain embodiments, the compositions may comprise a total amount of AHAs ranging from about 1 to about 20 wt %, about 1 to about 19 wt %, about 1 to about 18 wt %, about 1 to about 17 wt %, about 1 to about 16 wt %, about 1 to about 15 wt %, about 1 to about 14 wt %, about 1 to about 13 wt %, about 1 to about 12 wt %, about 1 to about 11 wt %, about 1 to about 10 wt %, about 1 to about 9 wt %, about 1 to about 8 wt %, about 1 to about 7 wt %, about 1 to about 6 wt %, about 1 to about 5 wt %, about 1 to about 4 wt %, about 1 to about 3 wt %, about 1 to about 2 wt %, about 2 to about 20 wt %, about 2 to about 19 wt %, about 2 to about 18 wt %, about 2 to about 17 wt %, about 2 to about 16 wt %, about 2 to about 15 wt %, about 2 to about 14 wt %, about 2 to about 13 wt %, about 2 to about 12 wt %, about 2 to about 11 wt %, about 2 to about 10 wt %, about 2 to about 9 wt %, about 2 to about 8 wt %, about 2 to about 7 wt %, about 2 to about 6 wt %, about 2 to about 5 wt %, about 2 to about 4 wt %, about 2 to about 3 wt %, about 3 to about 20 wt %, about 3 to about 19 wt %, about 3 to about 18 wt %, about 3 to about 17 wt %, about 3 to about 16 wt %, about 3 to about 15 wt %, about 3 to about 14 wt %, about 3 to about 13 wt %, about 3 to about 12 wt %, about 3 to about 11 wt %, about 3 to about 10 wt %, about 3 to about 9 wt %, about 3 to about 8 wt %, about 3 to about 7 wt %, about 3 to about 6 wt %, about 3 to about 5 wt %, about 3 to about 4 wt %, about 10 to about 20 wt %, about 10 to about 19 wt %, about 10 to about 18 wt %, about 10 to about 17 wt %, about 10 to about 16 wt %, about 10 to about 15 wt %, about 10 to about 14 wt %, about 10 to about 13 wt %, about 10 to about 12 wt %, about 11 to about 20 wt %, about 11 to about 19 wt %, about 11 to about 18 wt %, about 11 to about 17 wt %, about 11 to about 16 wt %, about 11 to about 15 wt %, about 11 to about 14 wt %, about 11 to about 13 wt %, about 12 to about 20 wt %, about 12 to about 19 wt %, about 12 to about 18 wt %, about 12 to about 17 wt %, about 12 to about 16 wt %, about 12 to about 15 wt %, about 12 to about 14 wt %, about 13 to about 20 wt %, about 13 to about 19 wt %, about 13 to about 18 wt %, about 13 to about 17 wt %, about 13 to about 16 wt %, or about 13 to about 15 wt %, including ranges and subranges therebetween, relative to the total weight of the composition in which it is present. In certain embodiments, the total amount of AHA ranges from about 2 to about 4 wt %, about 4 to about 7 wt %, or about 13 to about 16 wt %, relative to the total weight of the composition in which it is present. In other embodiments, the total amount of AHA is about 3 wt %, is between 5 and 6 wt %, or is between 14 and 15 wt %, relative to the total weight of the composition in which it is present.

In certain embodiments, compositions according to the disclosure comprise at least one BHA as a chemical peeling agent. For example, the compositions may comprise a total amount of BHAs ranging from about 0.01 to about 2 wt %, about 0.01 to about 1.9 wt %, about 0.01 to about 1.8 wt %, about 0.01 to about 1.7 wt %, about 0.01 to about 1.6 wt %, about 0.01 to about 1.5 wt %, about 0.01 to about 1.4 wt %, about 0.01 to about 1.3 wt %, about 0.01 to about 1.2 wt %, about 0.01 to about 1.1 wt %, about 0.01 to about 1.0 wt %, about 0.01 to about 0.9 wt %, about 0.01 to about 0.8 wt %, about 0.01 to about 0.7 wt %, about 0.01 to about 0.6 wt %, about 0.01 to about 0.5 wt %, about 0.01 to about 0.4 wt %, about 0.01 to about 0.3 wt %, about 0.01 to about 0.2 wt %, about 0.01 to about 0.1 wt %, about 0.05 to about 2 wt %, about 0.05 to about 1.9 wt %, about 0.05 to about 1.8 wt %, about 0.05 to about 1.7 wt %, about 0.05 to about 1.6 wt %, about 0.05 to about 1.5 wt %, about 0.05 to about 1.4 wt %, about 0.05 to about 1.3 wt %, about 0.05 to about 1.2 wt %, about 0.05 to about 1.1 wt %, about 0.05 to about 1.0 wt %, about 0.05 to about 0.9 wt %, about 0.05 to about 0.8 wt %, about 0.05 to about 0.7 wt %, about 0.05 to about 0.6 wt %, about 0.05 to about 0.5 wt %, about 0.05 to about 0.4 wt %, about 0.05 to about 0.3 wt %, about 0.05 to about 0.2 wt %, about 0.05 to about 0.1 wt %, about 0.1 to about 2 wt %, about 0.1 to about 1.9 wt %, about 0.1 to about 1.8 wt %, about 0.1 to about 1.7 wt %, about 0.1 to about 1.6 wt %, about 0.1 to about 1.5 wt %, about 0.1 to about 1.4 wt %, about 0.1 to about 1.3 wt %, about 0.1 to about 1.2 wt %, about 0.1 to about 1.1 wt %, about 0.1 to about 1.0 wt %, about 0.1 to about 0.9 wt %, about 0.1 to about 0.8 wt %, about 0.1 to about 0.7 wt %, about 0.1 to about 0.6 wt %, about 0.1 to about 0.5 wt %, about 0.1 to about 0.4 wt %, about 0.1 to about 0.3 wt %, or about 0.1 to about 0.2 wt %, including ranges and subranges therebetween, relative to the total weight of the composition in which it is present. In certain embodiments, the total amount of BHA is about 0.05 wt %, about 0.06 wt %, about 0.07 wt %, about 0.08 wt %, about 0.09 wt %, about 0.1 wt %, about 0.11 wt %, about 0.12 wt %, about 0.13 wt %, about 0.14 wt %, about 0.15 wt %, about 0.16 wt %, about 0.17 wt %, about 0.18 wt %, about 0.19 wt %, about 0.2 wt %, about 0.21 wt %, about 0.22 wt %, about 0.23 wt %, about 0.24 wt %, or about 0.25 wt %, relative to the total weight of the composition in which it is present.

In certain embodiments, compositions according to the disclosure comprise trichloroacetic acid as a chemical peeling agent. For example, the compositions may comprise a total amount of trichloroacetic acid ranging from about 1 to about 25 wt %, about 1 to about 24 wt %, about 1 to about 23 wt %, about 1 to about 22 wt %, about 1 to about 21 wt %, about 1 to about 20 wt %, about 1 to about 19 wt %, about 1 to about 18 wt %, about 1 to about 17 wt %, about 1 to about 16 wt %, about 1 to about 15 wt %, about 1 to about 14 wt %, about 1 to about 13 wt %, about 1 to about 12 wt %, about 1 to about 11 wt %, about 1 to about 10 wt %, about 1 to about 9 wt %, about 1 to about 8 wt %, about 1 to about 7 wt %, about 1 to about 6 wt %, about 1 to about 5 wt %, about 1 to about 4 wt %, about 1 to about 3 wt %, about 1 to about 2 wt %, about 3 to about 25 wt %, about 3 to about 24 wt %, about 3 to about 23 wt %, about 3 to about 22 wt %, about 3 to about 21 wt %, about 3 to about 20 wt %, about 3 to about 19 wt %, about 3 to about 18 wt %, about 3 to about 17 wt %, about 3 to about 16 wt %, about 3 to about 15 wt %, about 3 to about 14 wt %, about 3 to about 13 wt %, about 3 to about 12 wt %, about 3 to about 11 wt %, about 3 to about 10 wt %, about 3 to about 9 wt %, about 3 to about 8 wt %, about 3 to about 7 wt %, about 3 to about 6 wt %, about 3 to about 5 wt %, 5 to about 25 wt %, about 5 to about 24 wt %, about 5 to about 23 wt %, about 5 to about 22 wt %, about 5 to about 21 wt %, about 5 to about 20 wt %, about 5 to about 19 wt %, about 5 to about 18 wt %, about 5 to about 17 wt %, about 5 to about 16 wt %, about 5 to about 15 wt %, about 5 to about 14 wt %, about 5 to about 13 wt %, about 5 to about 12 wt %, about 5 to about 11 wt %, about 5 to about 10 wt %, about 5 to about 9 wt %, about 5 to about 8 wt %, about 5 to about 7 wt %, 8 to about 25 wt %, about 8 to about 24 wt %, about 8 to about 23 wt %, about 8 to about 22 wt %, about 8 to about 21 wt %, about 8 to about 20 wt %, about 8 to about 19 wt %, about 8 to about 18 wt %, about 8 to about 17 wt %, about 8 to about 16 wt %, about 8 to about 15 wt %, about 8 to about 14 wt %, about 8 to about 13 wt %, about 8 to about 12 wt %, about 8 to about 11 wt %, about 8 to about 10 wt %, about 10 to about 25 wt %, about 10 to about 24 wt %, about 10 to about 23 wt %, about 10 to about 22 wt %, about 10 to about 21 wt %, about 10 to about 20 wt %, about 10 to about 19 wt %, about 10 to about 18 wt %, about 10 to about 17 wt %, about 10 to about 16 wt %, about 10 to about 15 wt %, about 10 to about 14 wt %, about 10 to about 13 wt %, about 10 to about 12 wt %, 12 to about 25 wt %, about 12 to about 24 wt %, about 12 to about 23 wt %, about 12 to about 22 wt %, about 12 to about 21 wt %, about 12 to about 20 wt %, about 12 to about 19 wt %, about 12 to about 18 wt %, about 12 to about 17 wt %, about 12 to about 16 wt %, about 12 to about 15 wt %, or about 12 to about 14 wt %, relative to the total weight of the composition in which it is present.

Optionally, the compositions may comprise more than one peeling agent, such as, for example, at least two peeling agents, at least three peeling agents, at least four peeling agents, and so on. For example, the compositions may comprise two or more AHAs, may comprise two or more BHAs, may comprise a combination of one or more AHAs and one or more BHAs, and so on. By way of non-limiting example only, the compositions may comprise glycolic acid and lactic acid, may comprise glycolic acid and salicylic acid, may comprise lactic acid and salicylic acid, may comprise glycolic acid and trichloroacetic acid, may comprise lactic acid and trichloroacetic acid, may comprise salicylic acid and trichloroacetic acid, may comprise glycolic acid, lactic acid, and trichloroacetic acid, may comprise glycolic acid, salicylic acid, and trichloroacetic acid, and so on.

In various embodiments, compositions according to the disclosure comprise at least one peeling agent but are free or substantially free of acetyl trifluoromethylphenyl valylglycine.

By way of non-limiting example only, compositions comprising at least one peeling agent that are free or substantially free of acetyl trifluoromethylphenyl valylglycine may comprise additional components and amounts thereof typically used in peeling compositions, such as, for example, at least one solvent, at least one oil, and additional optional components. Such compositions may, in certain embodiments, have a pH not less than about 3.0, such as, for example, not less than about 3.5. The pH can be adjusted to the desired value by addition of a base (organic or inorganic) to the composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid such as a carboxylic acid, for example citric acid.

For example, the compositions comprising at least one peeling agent that are free or substantially free of acetyl trifluoromethylphenyl valylglycine may comprise at least one solvent chosen from water and organic solvents. By way of example, the compositions may comprise water in an amount of at least about 15 wt %, such as, for example, from about 20 wt % to about 70 wt %, from about 25 wt % to about 60 wt %, from about 30 wt % to about 50 wt %, or from about 35 wt % to about 40 wt %, including all ranges and subranges thereof.

In further embodiments, the compositions comprising at least one peeling agent that are free or substantially free of acetyl trifluoromethylphenyl valylglycine include one or more organic solvents such as monoalcohols and polyols, for example ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. In some embodiments, the alcohol solvent is selected from ethanol, isopropyl alcohol, ethylene glycol, propylene glycol, butylene glycol, hexylene glycol, propanediol, glycerin, or mixtures thereof. Optionally, in some embodiments the organic solvent(s) may be present in amounts up to about 20 wt %, such as from about 0.1 to about 20 wt %, from about 1 to about 20 wt %, from about 5 to about 20 wt %, or from about 10 to about 15 wt %, based on the weight of the composition.

In various embodiments, the compositions comprising at least one peeling agent that are free or substantially free of acetyl trifluoromethylphenyl valylglycine include one or more oils. Optionally, the at least one oil may be immiscible in water. For example, the at least one oil may be selected from hydrocarbons, silicones, fatty alcohols, glycols, and vegetable oils. The at least one oil may include one or a combination of polar and non-polar oil. In some embodiments, the oil may be chosen from hydrocarbon-based oils from plants or of plant origin, mineral oil, ester oils, fatty alcohols containing from 12 to 26 carbon atoms, fatty acids containing from 12 to 26 carbon atoms and vinylpyrrolidone copolymers, and mixtures thereof. In some particular embodiments, an oil may be chosen from purcellin oil (cetostearyl octanoate), squalane, hemisqualane, isononyl isononanoate, C12 to C15 alkyl benzoate, 2-ethylhexyl palmitate, isodecyl neopentanoate, tridecyl neopentanoate, octyldodecyl neopentanoate, 2-octyldodecyl stearate, 2-octyldodecyl erucate, oleyl erucate, isostearyl isostearate, 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate, 2-octyldodecyl myristate and 2-diethylhexyl succinate, cocoglyceride, cyclomethicone, dimethicone, dicaprylyl carbonate, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C9-C15 alcohols, alkanes, mineral oil, silicone, dimethyl polysiloxane, ether, polyoxypropylene butyl ether, polyoxypropylene cetyl ether, C12-C15 alkyl benzoate, aryl alkyl benzoate, Isopropyl Lauroyl sarcosinate, and any combinations thereof.

In some embodiments, the oil is chosen from C13-15 alkane (hemisqualane), dicaprylyl carbonate, isononyl isononanoate, isopropyl myristate, dimethicone, squalane, mineral oil, or mixtures thereof. In one exemplary and non-limiting embodiment, the oil comprises one or a combination of C13-15 alkane (hemisqualane), dicaprylyl carbonate, isononyl isononanoate, isopropyl myristate, dimethicone, squalane, and mineral oil. In some embodiments, the oil includes isopropyl myristate.

In accordance with the various embodiments, the amount of at least one oil present in the compositions comprising at least one peeling agent that are free or substantially free of acetyl trifluoromethylphenyl valylglycine is at least about 5 wt %, at least about 10 wt %, at least about 15 wt %, or at least about 20 wt %, for example can range from about 5 wt % to about 70 wt %, from about 10 wt % to about 65 wt %, from about 15 wt % to about 60 wt %, or from about 20 wt % to about 55 wt %, including all ranges and subranges thereof, based on the weight of the composition. In one embodiment, the total amount of oil ranges from about 20 wt % to about 60 wt % of the composition.

The compositions comprising at least one peeling agent that are free or substantially free of acetyl trifluoromethylphenyl valylglycine can also comprise one or more additional optional components typically used in chemical peel compositions, for example one or more additives chosen from citric acid, sodium chloride, chelating agents, antimicrobial agents, neutralizing agents, vitamins (e.g. tocopherol), fragrances, pearlescent agents, odor absorbers, coloring materials, essential oils, fruit extracts, or combinations thereof.

In various embodiments, additional optional components may include one or a combination of antimicrobial agents and their salts, selected from, for example, chlorphenesin, caprylyl glycol, phenoxyethanol, caprylhydroxamic acid, benzoic acid, salicylic acid, benzyl alcohol, phenethyl alcohol, benzalkonium chloride, 4-hydroxyacetophenone, piroctone olamine, hexyl glycerin, ethylhexylglycerin, octylglycerin, benzylglycerin, 3-heptoyl-2,2-propandiol, 1,2-hexandiol, pentylene glycol, or mixtures thereof.

In various embodiments, the amount of one or more additional optional components, alone or in combination, may be present in the compositions comprising at least one chemical peeling agent that are free or substantially free of acetyl trifluoromethylphenyl valylglycine in an amount ranging from about 0.001 wt % to about 20 wt %, such as from about 0.005 wt % to about 0.01 wt %, from about 0.01 wt % to about 0.1 wt %, from about 0.15 wt % to about 5 wt %, from about 0.40 wt % to about 4 wt %, from about 0.5 wt % to about 2.5 wt % by weight, or from about 1 wt % to about 2 wt %, including all ranges and subranges thereof, based on the weight of the composition.

### II. Methods

Methods according to the disclosure comprise applying (a) acetyl trifluoromethylphenyl valylglycine and (b) at least one peeling agent to the skin. When used together in the methods described herein, the acetyl trifluoromethylphenyl valylglycine and peeling agent(s) surprisingly work together to increase, accelerate, and/or improve skin renewal, and/or improve the appearance of skin.

The skin that can be treated may, for example, be on the face, neck, chest, or hands. It should be understood that the skin to be treated according to methods disclosed herein, e.g. the skin to which the acetyl trifluoromethylphenyl valylglycine and peeling agent(s) are applied, is substantially the same area of the skin. Thus, for example, a method of treating skin according to various embodiments may include a method of applying both acetyl trifluoromethylphenyl valylglycine and one or more peeling agent(s) to the face, which applications may be simultaneous or consecutive. Thus, in various embodiments, the acetyl trifluoromethylphenyl valylglycine and the at least one peeling agent can be applied to the skin simultaneously (e.g. when both are present in one composition or when both are present in two compositions that are mixed at or near the time of use), substantially simultaneously, or consecutively (e.g. when each is present in a separate composition, which are applied sequentially to the skin, or when a non-chemical peeling agent is used).

In certain embodiments, the methods comprise applying an amount of at least one chemical peeling agent such that about 0.1 gram or more of the at least one chemical peeling agent is applied to the skin. For example, the skin treatment composition may be applied such that about 0.05 gram per decimeter (g/dm²) or more, such as about 0.06 g/dm² or more, such as about 0.07 g/dm² or more, such as about 0.08 g/dm² or more, such as about 0.09 g/dm² or more, such as about 0.1 g/dm² or more, such as about 0.15 g/dm² or more, about 0.2 g/dm² or more, about 0.3 g/dm² or more, about 0.4 g/dm² or more, about 0.5 g/dm² or more, about 0.75 g/dm² or more, about 1 g/dm² or more, about 1.5 g/dm² or more, about 2 g/dm² or more, about 2.5 g/dm² or more, or about 3 g/dm² or more of chemical peeling agent(s) is applied to the skin. In some embodiments, the composition is applied such that about 0.05 to about 10 g/dm², about 0.06 to about 9 g/dm², about 0.07 to about 8 g/dm², about 0.08 to about 7 g/dm², about 0.09 to about 6 g/dm², about 0.1 to about 5 g/dm², about 0.15 to about 4 g/dm², about 0.2 to about 3 g/dm², about 0.3 to about 2 g/dm², about 0.4 to about 1 g/dm²; about 0.5 to about 10 gram per decimeter (g/dm²), about 0.5 to about 9 g/dm², about 0.5 to about 8 g/dm², about 0.5 to about 7 g/dm², about 0.5 to about 6 g/dm², about 0.5 to about 5 g/dm², about 0.5 to about 4 g/dm², about 0.5 to about 3 g/dm², about 0.5 to about 2 g/dm², about 0.5 to about 1 g/dm²; or about 1 to about 10 gram per decimeter (g/dm²), about 1 to about 9 g/dm², about 1 to about 8 g/dm², about 1 to about 7 g/dm², about 1 to about 6 g/dm², about 1 to about 5 g/dm², about 1 to about 4 g/dm², about 1 to about 3 g/dm², or about 1 to about 2 g/dm² of chemical peeling agent(s) is applied to the skin.

In certain embodiments, the methods comprise applying an amount of acetyl trifluoromethylphenyl valylglycine such that about 0.1 gram or more of acetyl trifluoromethylphenyl valylglycine is applied to the skin. For example, the skin treatment composition may be applied such that about 0.05 gram per decimeter (g/dm²) or more, such as about 0.06 g/dm² or more, such as about 0.07 g/dm² or more, such as about 0.08 g/dm² or more, such as about 0.09 g/dm² or more, such as about 0.1 g/dm² or more, such as about 0.15 g/dm² or more, about 0.2 g/dm² or more, about 0.3 g/dm² or more, about 0.4 g/dm² or more, about 0.5 g/dm² or more, about 0.75 g/dm² or more, about 1 g/dm² or more, about 1.5 g/dm² or more, about 2 g/dm² or more, about 2.5 g/dm² or more, or about 3 g/dm² or more of acetyl trifluoromethylphenyl valylglycine is applied to the skin. In some embodiments, the composition is applied such that about 0.05 to about 10 g/dm², about 0.06 to about 9 g/dm², about 0.07 to about 8 g/dm², about 0.08 to about 7 g/dm², about 0.09 to about 6 g/dm², about 0.1 to about 5 g/dm², about 0.15 to about 4 g/dm², about 0.2 to about 3 g/dm², about 0.3 to about 2 g/dm², about 0.4 to about 1 g/dm²; about 0.5 to about 10 gram per decimeter (g/dm²), about 0.5 to about 9 g/dm², about 0.5 to about 8 g/dm², about 0.5 to about 7 g/dm², about 0.5 to about 6 g/dm², about 0.5 to about 5 g/dm², about 0.5 to about 4 g/dm², about 0.5 to about 3 g/dm², about 0.5 to about 2 g/dm², about 0.5 to about 1 g/dm²; or about 1 to about 10 gram per decimeter (g/dm²), about 1 to about 9 g/dm², about 1 to about 8 g/dm², about 1 to about 7 g/dm², about 1 to about 6 g/dm², about 1 to about 5 g/dm², about 1 to about 4 g/dm², about 1 to about 3 g/dm², or about 1 to about 2 g/dm² of acetyl trifluoromethylphenyl valylglycine is applied to the skin.

In some embodiments, at least one chemical peeling agent can be left on the skin for at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours, such as between 10 seconds and 48 hours, before the at least one chemical peel is removed, such as, e.g., by rinsing. In an embodiment, the at least one chemical peeling agent is not removed prior to application of one or more other skin compositions, such as, e.g., an acetyl trifluoromethylphenyl valylglycine comprising composition, moisturizers, sunscreen, or other anti-aging treatments.

The at least one peeling agent can, in some embodiments, be applied to the skin at least one time a day, such as at least two times a day.

The at least one peeling agent can be applied in any manner known, and will vary depending on the type of peeling agent chosen. For example, chemical peeling agents may be applied directly to the skin (e.g. with the fingers) or using an implementation such as a ball or a pad particularly adapted for surface wiping. The pad may include an absorbent core having the ability to absorb and retain liquid material, and a liner layer in contact with and covering at least one side of the absorbent core. The liner layer has the ability to retain and wick liquid material through the liner layer. The pad may be fabricated from any convenient material, where suitable materials of interest include, but are not limited to cellulosic materials, polymeric materials, etc. The wipe can be formed of a paper, cotton or textile-based substrate. In some instances, the absorbent pad material is made up of hydrophilic materials. In other embodiments, absorbent patches are selected from the group consisting of gauzes, cellulosic pads, agarose gels, acrylamide gels, or hydrogels.

The composition comprising the chemical peeling agent(s) can be applied directly to the skin or using a device such as a ball or pad, such as a cotton ball or a cotton pad. The ball or pad can be soaked with about 0.5 mL to about 10 mL of a composition comprising the at least one chemical peel, such as about 0.7 mL to about 9 mL, about 0.9 mL to about 8 mL, about 1 mL to about 7 mL, about 1.5 mL to about 6 mL, about 2 mL to about 5 mL, about 2 mL to about 4 mL, about 2 mL to about 3 mL, about 3 mL to about 5 mL, or about 4 mL to about 5 mL of a composition comprising the peeling agent(s). When using a pad or ball, the peeling agent(s) can be applied by making at least 1 pass of the ball or pad over the skin, such as at least 2 passes, at least 3 passes, at least 4 passes, at least 5 passes, at least 6 passes, at least 7 passes, at least 8 passes, at least 9 passes, at least 10 passes, such as between 1 and 10 passes, 1 and 9 passes, 1 and 8 passes, 1 and 7 passes, 1 and 6 passes, 1 and 5 passes, 1 and 4 passes, 1 and 3 passes, and 1 and 2 passes.

Physical or non-chemical peeling agents can be applied in any manner such physical peeling agents are typically used to effect skin peeling or exfoliation. Therefore, it is to be understood that the language “applying at least one peeling agent to the skin” and variations thereof in embodiments where a non-chemical (physical) peeling agent such as microbeads or another physical peeling agent present in a composition is used, it is meant to convey using the composition in a manner that exfoliates the skin, e.g. by applying the composition on to the skin and rubbing the skin to effect exfoliation by the microbeads, etc. Similarly, in embodiments where a non-chemical (physical) peeling agent such as an exfoliating sponge, pad, etc. is used, the language “applying at least one peeling agent to the skin” and variations thereof is meant to convey that the skin is treated in a manner that exfoliates the skin, e.g. by scrubbing the skin with the exfoliating sponge, pad, etc.

If the (a) acetyl trifluoromethylphenyl valylglycine and (b) at least one peeling agent are applied consecutively, the methods disclosed herein may include, in various embodiments, applying acetyl trifluoromethylphenyl valylglycine to the skin within about 48 hours before or after applying the at least one peeling agent to the skin. For example, in some embodiments the acetyl trifluoromethylphenyl valylglycine may be applied within about 24 hours, within about 20 hours, within about 16 hours, within about 12 hours, within about 10 hours, within about 8 hours, within about 6 hours, within about 4 hours, within about 2 hours, within about 1 hour, within about 30 minutes, within about 20 minutes, within about 10 minutes, within about 5 minutes, or within about 1 minute after the application of the at least one peeling agent to the skin. In other embodiments, the acetyl trifluoromethylphenyl valylglycine may be applied within about 24 hours, within about 20 hours, within about 16 hours, within about 12 hours, within about 10 hours, within about 8 hours, within about 6 hours, within about 4 hours, within about 2 hours, within about 1 hour, within about 30 minutes, within about 20 minutes, within about 10 minutes, within about 5 minutes, or within about 1 minute before the application of the at least one peeling agent to the skin.

In an embodiment, the acetyl trifluoromethylphenyl valylglycine can be left on the skin for at most 10 seconds, at most 20 seconds, at most 30 seconds, at most 40 seconds, at most 50 seconds, at most 1 minute, at most 2 minutes, at most 3 minutes, at most 5 minutes, at most 10 minutes, at most 15 minutes, at most 20 minutes, at most 25 minutes, at most 30 minutes, at most 35 minutes, at most 40 minutes, at most 45 minutes, at most 1 hour, at most 2 hours, at most 5 hours, at most 8 hours, at most 10 hours, at most 15 hours, at most 20 hours, at most 24 hours, at most 36 hours, or at most 48 hour before the acetyl trifluoromethylphenyl valylglycine is removed such as by, e.g., rinsing. In an embodiment, the acetyl trifluoromethylphenyl valylglycine is not removed prior to application of other skin compositions, such as, e.g, a composition comprising a peeling agent, moisturizers, sunscreen, or other anti-aging treatments.

The acetyl trifluoromethylphenyl valylglycine can be applied at least one time a day, at least two times a day, at least three times a day, at least four times a day, or at least five times a day, such as from one to five times a day, from one to four times a day, from one to three times a day, or from one to two times a day.

In some embodiments, the at least one chemical peeling agent is left on the skin for at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours before the at least one chemical peeling agent is removed, such as by, e.g., rinsing. In an embodiment, the at least one chemical peeling agent is left on the skin for at most 10 seconds, at most 20 seconds, at most 30 seconds, at most 40 seconds, at most 50 seconds, at most 1 minute, at most 2 minutes, at most 3 minutes, at most 5 minutes, at most 10 minutes, at most 15 minutes, at most 20 minutes, at most 25 minutes, at most 30 minutes, at most 35 minutes, at most 40 minutes, at most 45 minutes, at most 1 hour, at most 2 hours, at most 5 hours, at most 8 hours, at most 10 hours, at most 15 hours, at most 20 hours, at most 24 hours, at most 36 hours, or at most 48 hour before the at least one chemical peeling agent is removed, such as by, e.g., rinsing. For example, the at least one chemical peeling agent may be left on the skin for a period of time ranging from 5 minutes to 60 minutes, such as from 5 minutes to 50 minutes, from 5 minutes to 40 minutes, from 5 minutes to 35 minutes, from 5 minutes to 30 minutes, from 5 minutes to 25 minutes, from 5 minutes to 20 minutes, from 5 minutes to 15 minutes, or from 5 minutes to 10 minutes before the at least one chemical peeling agent is removed. As a further example, the at least one chemical peeling agent may be left on the skin for a period of time ranging from 10 minutes to 60 minutes, such as from 10 minutes to 50 minutes, from 10 minutes to 40 minutes, from 10 minutes to 35 minutes, from 10 minutes to 30 minutes, from 10 minutes to 25 minutes, from 10 minutes to 20 minutes, or from 10 minutes to 15 minutes before the at least one chemical peeling agent is removed.

The methods may, in some embodiments, include applying the acetyl trifluoromethylphenyl valylglycine to the skin more than one time, e.g. before and after the application of the at least one peeling agent to the skin. The methods may, in some embodiments, include applying the at least one peeling agent to the skin more than one time, e.g. before and after the application of the acetyl trifluoromethylphenyl valylglycine to the skin. In some cases, the method may be repeated to achieve optimal results.

If applied simultaneously, the acetyl trifluoromethylphenyl valylglycine and the peeling agent(s) may optionally be present in a single composition. Alternatively, the acetyl trifluoromethylphenyl valylglycine and the peeling agent(s) may optionally be present in separate compositions, e.g. two or more compositions, three or more compositions, etc., which can be mixed at or near the time of use.

The compositions comprising acetyl trifluoromethylphenyl valylglycine and/or at least one peeling agent can be, for example, topical skin treatment composition(s). The topical skin treatment composition(s) can be formulated as a cream, a lotion, a serum, a gel, or an ampoule, or any other topically suitable form. For example, the skin treatment compositions may be in the form of water-in-oil emulsions, oil-in-water emulsions, silicone-in-water emulsions, water-in-silicone emulsions, or combinations thereof. The skin treatment compositions may be used in methods for increasing or improving skin renewal.

The acetyl trifluoromethylphenyl valylglycine can be applied to the skin at least one time per day, for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84 consecutive or non-consecutive days, preferably consecutive days. The acetyl trifluoromethylphenyl valylglycine may be applied to the skin at least one time per day, for at least 1, 2, 3, 4, 5, 6, or 7 days per week, preferably at least 2 times per day. For Example, the acetyl trifluoromethylphenyl valylglycine may be applied to the skin at least two times per day, wherein the second application is at least about 6 hours, preferably at least about 8 hours, more preferably at least about 10 hours, most preferably at least about 12 hours after the first application.

The at least one peeling agent, e.g. a physical or chemical peeling agent, can be applied to the skin at least one time per day for at least 1, 2, 3, 4, 5, 6, 7, 8, 9, 10, 11, 12, 13, 14, 15, 16, 17, 18, 19, 20, 21, 22, 23, 24, 25, 26, 27, or 28 consecutive or non-consecutive days, preferably nonconsecutive days. The at least one peeling agent may be applied to the skin at least one time per day for at least 1, 2, 3, 4, 5, 6, or 7 days a week. For example, the at least one peeling agent can be applied to the skin at least every other day.

The at least one peeling agent may be applied to the skin at least every other day fora period of at least a 1, 2, 3, 4, 5, 6, 7, 8, 9, 10, 11, 12, 13, 14, 15, 16, 17, 18, 19, 20, 21, 22, 23, 24, 25, 26, 27, or 28 days, followed by a period of at least a 1, 2, 3, 4, 5, 6, 7, 8, 9, 10, 11, 12, 13, 14 days wherein the peeling agent is not applied.

Alternatively, the acetyl trifluoromethylphenyl valylglycine may be present in a composition and the skin may be treated with the acetyl trifluoromethylphenyl valylglycine and a non-chemical peeling agent sequentially, in the manner described herein for treatment with acetyl trifluoromethylphenyl valylglycine and chemical peeling agents.

It may be advantageous in some embodiments to alternate treatment periods where skin is treated with the combination of acetyl trifluoromethylphenyl valylglycine and peeling agent(s) as described herein with rest periods, for example when the skin is not treated with either acetyl trifluoromethylphenyl valylglycine or peeling agent(s), or when skin is being treated with either the acetyl trifluoromethylphenyl valylglycine or the peeling agent(s) but not both. For example, in some embodiments a regimen may include a treatment period of at least one week, at least two weeks, at least three weeks, at least four weeks, at least one month, at least two months, etc., wherein the skin is treated by applying acetyl trifluoromethylphenyl valylglycine and at least one peeling agent according to the methods described herein, followed by a rest period where only acetyl trifluoromethylphenyl valylglycine is applied to the skin. The rest period may, in various embodiments, be equal or approximately equal to, longer than, or shorter than the treatment period. In some embodiments, the rest period may be about the same as, about one-half, about one-third, or about one-quarter the length of the treatment period. For example, one regimen may include a treatment period of about 2 weeks followed by a rest period of about 2 weeks. Another exemplary regimen may include a treatment period of about 4 weeks followed by a rest period of about 2 weeks. Yet another exemplary regimen may include a treatment period of about 2 weeks followed by a rest period of about 1 week. A still further exemplary regimen may include a treatment period of about 2 months followed by a rest period of about 4 weeks.

It has been surprisingly and unexpectedly discovered that the use of acetyl trifluoromethylphenyl valylglycine with at least one peeling agent improved skin turnover. Therefore, in various exemplary and non-limiting embodiments, the methods relate to improving skin turnover, comprising applying acetyl trifluoromethylphenyl valylglycine and at least one chemical peeling agent simultaneously or sequentially, as described above.

In exemplary and non-limiting embodiments, the methods disclosed herein may comprise a regimen comprising applying to the skin:
(a) acetyl trifluoromethylphenyl valylglycine at least one time per day; and
(b) at least one peeling agent at least one time every two to three days,
wherein the regimen lasts for a period of time of at least about 7 days, such as at least about 10 days, at least about 14 days, at least about 21 days, or at least about 28 days.

In further exemplary and non-limiting embodiments, the methods disclosed herein may comprise a regimen for treating skin, the regimen comprising applying to the skin:
(a) a composition comprising acetyl trifluoromethylphenyl valylglycine, at least one time per day; and
(b) a composition comprising at least one peeling agent, at least one time every two to three days,
wherein the regimen lasts for a period of time of at least about 7 days, such as at least about 10 days, at least about 14 days, at least about 21 days, or at least about 28 days.

In yet further exemplary and non-limiting embodiments, the methods disclosed herein may comprise a regimen for treating skin, the regimen comprising applying to the skin:
(a) a composition comprising acetyl trifluoromethylphenyl valylglycine, at; and
(c) a composition comprising at least one peeling agent, at least one time every two to three days,
wherein the regimen lasts for a period of time of at least about 7 days, such as at least about 10 days, at least about 14 days, at least about 21 days, or at least about 28 days.

In still further exemplary and non-limiting embodiments, the methods disclosed herein may comprise a regimen for treating skin, the regimen comprising applying to the skin:
(a) from about 0.1 to about 10 wt % of acetyl trifluoromethylphenyl valylglycine, at least one time, preferably at least two times, per day; and
(b) optionally from about 0.1 to about 25 wt % of at least one chemical peeling agent chosen from α-hydroxy acids, β-hydroxy acids, or mixtures thereof, at least one time every two to three days,
wherein the regimen lasts for a period of time of at least about 7 days, such as at least about 10 days, at least about 14 days, at least about 21 days, or at least about 28 days.

### III. Kits

The disclosure also relates to kits comprising the synergistic combination of acetyl trifluoromethylphenyl valylglycine and at least one peeling agent. For example, in some embodiments, the kits comprise acetyl trifluoromethylphenyl valylglycine and at least one chemical peeling agent. In further embodiments, the kits comprise acetyl trifluoromethylphenyl valylglycine and at least one composition comprising a non-chemical peeling agent, such as a composition comprising microbeads. In still further embodiments, the kits comprise acetyl trifluoromethylphenyl valylglycine and at least one exfoliating implementation, such as, for example, a sponge or pad for exfoliating the skin which may or may not comprise a chemical peeling agent.

The kits may comprise at least two containers or compartments, where a first container or compartment comprises (a) a composition comprising acetyl trifluoromethylphenyl valylglycine, and a second container or compartment comprises (b) at least one peeling agent, for example a composition comprising at least one peeling agent and/or at least one exfoliating implementation. The kits may optionally further comprise at least one additional container or compartment.

By way of non-limiting example only, the kits may be in the form of a dual-chamber container with a dispenser. The first and second chambers may be separate such that compositions (a) and (b) are segregated from each other prior to use. The dual-chamber container may optionally comprise a doser for mixing and dispensing amounts of compositions (a) and (b) such that compositions (a) and (b) are dispensed as a single composition comprising the desired amounts of acetyl trifluoromethylphenyl valylglycine and peeling agent(s), e.g. chemical peeling agent(s), for use in various methods according to the disclosure.

As a further non-limiting example, the kits may comprise a first container with a dispenser, wherein the first container comprises (a) a composition comprising acetyl trifluoromethylphenyl valylglycine, and a second container with a dispenser, wherein the second container comprises (b) a composition comprising at least one peeling agent, e.g. at least one chemical peeling agent. Optionally, the kits may further comprise instructions providing dosage, order, and timing of application, etc., of compositions (a) and (b) for use in various methods according to the disclosure.

Having described various embodiments of the present disclosure in detail, it will be apparent that modifications and variations thereof are possible without departing from the scope of the disclosure. Furthermore, it should be appreciated that all examples in the present disclosure, while illustrating embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Thus, where the disclosure refers to an element “chosen from A, B, C, D, E, F, and/or mixtures thereof,” it indicates that that one of A, B, C, D, and F may be included (e.g. only A, only B, only C, etc.), or a mixture of any two of A, B, C, D, E, and F may be included (e.g. a mixture of A and B, a mixture of A and C, a mixture of C, D, and E, etc.).

The term “and/or” should be understood to include both the conjunctive and the disjunctive. For example, “one or more chemical and/or physical peeling agents” means “one or more chemical peeling agents and one or more physical peeling agents” as well as “one or more chemical peeling agents or one or more physical peeling agents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” “or a combination thereof,” and variations thereof are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

Unless otherwise indicated, all percentages herein are by weight, relative to the total weight of the composition.

All percentages, parts, and ratios herein are based upon the total weight of the compositions of the present disclosure, unless otherwise indicated.

As used herein, the term “treat” (and its grammatical variations, such as “treating”) refers to the application of the compositions of the present disclosure onto the surface of the skin.

As used herein, the terms “skin turnover,” “epidermal turnover,” “skin renewal” and “epidermal renewal” are used interchangeably to refer to the skin's process for turning over skin cells, including the formation of new cells and/or the shedding of old cells.

As used herein, the term “day” is intended to cover a period of twenty-four (24) consecutive hours, which may be, but is not necessarily, a calendar day. For example, applying a composition to the skin one time per day means that after the composition is applied to the skin, the composition is not applied to the skin again until after 24 consecutive hours from the time of the application pass. Similarly, applying a composition to the skin two times per day means that after the composition is first applied to the skin, the composition is applied to the skin again one time within the next 24 consecutive hours and then not applied to the skin again until after 24 consecutive hours from the time of the first application pass.

As used herein, the term “week” is intended to cover a period of seven (7) consecutive days (i.e. 7 consecutive 24-hour periods), which may be, but is not necessarily, a calendar week. For example, applying a composition to the skin one time per week means that after the composition is applied to the skin, the composition is not applied to the skin again until after 7 consecutive days from the time of the application pass. Similarly, applying a composition to the skin two times per week means that after the composition is first applied to the skin, the composition is applied to the skin again one time within the next 7 consecutive days and then not applied to the skin again until after 7 consecutive days from the time of the first application pass.

The terms “substantially without,” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “substantially free” or “essentially free” as used herein means the specific material may be present in small amounts that do not materially affect the basic and novel characteristics of the compositions according to the disclosure. For instance, there may be less than 2 wt % by weight of a specific material added to a composition, based on the total weight of the compositions (provided that an amount of less than 2 wt % by weight does not materially affect the basic and novel characteristics of the compositions according to the disclosure. Similarly, the compositions may include less than 2 wt %, less than 1.5 wt %, less than 1 wt %, less than 0.5 wt %, less than 0.1 wt %, less than 0.05 wt %, or less than 0.01 wt %, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point. Thus, a range of “1 wt % to 10 wt %, such as 2 wt % to 8 wt %, such as 3 wt % to 5 wt %,” is intended to encompass ranges of “1 wt % to 8 wt %,” “1 wt % to 5 wt %,” “2 wt % to 10 wt %,” and so on. All numbers, amounts, ranges, etc., are intended to be modified by the term “about,” whether or not so expressly stated. Similarly, a range given of “about 1 wt % to 10 wt %” is intended to have the term “about” modifying both the 1 wt % and the 10 wt % endpoints. The term “about” is used herein to indicate a difference of up to +/−10 wt % from the stated number, such as +/−9 wt %, +/−8 wt %, +/−7 wt %, +/−6 wt %, +/−5 wt %, +/−4 wt %, +/−3 wt %, +/−2 wt %, or +/−1 wt %. Likewise, all endpoints of ranges are understood to be individually disclosed, such that, for example, a range of 1:2 to 2:1 is understood to disclose a ratio of both 1:2 and 2:1.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

Implementation of the present disclosure is provided by way of the following examples. The examples serve to illustrate the technology without being limiting in nature.

### Example 1

### Effect on Elastase Activity

The ability of acetyl trifluoromethylphenyl valylglycine (“ATV”), glycolic acid, and/or lactic acid to inhibit the elastin-hydrolyzing effect of elastase was evaluated in a Neutrophil Elastase (NE) Inhibition in tubo assay (MAK213, Sigma-Aldrich, MO). Neutrophil Elastase activity was measured by hydrolyzing the substrate to yield a fluorescent product (λex=400/λem=505 nm) proportional to the enzymatic activity present.

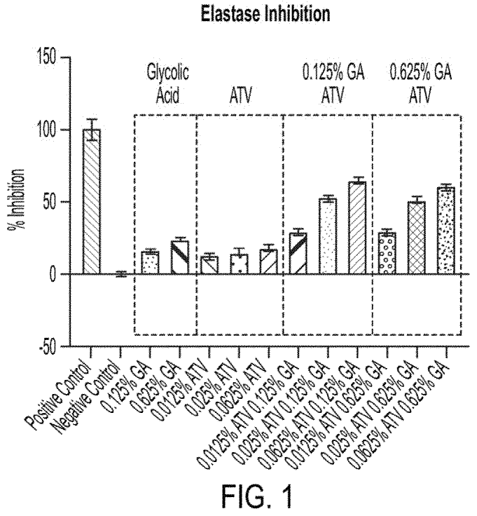

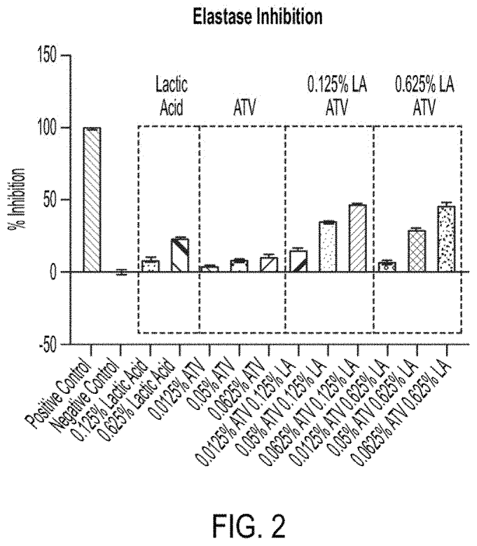

A 4× sample inhibitor solution of the each tested chemical peeling agent and acetyl trifluoromethylphenyl valylglycine, and every combination provided in FIGS. 1 and 2 was prepared. The inhibitor/positive control (Elastase Inhibitor, SPCK, AG Scientific), enzyme/negative control (contains elastase), neutrophil elastase, and substrate solutions were prepared. The test compounds, controls, and neutrophil elastase were added to a 96-well plate, protected from light, and incubated at 37° C. for 5 minutes. The elastin substrate was then added to each well and the fluorescence (FLU, λex=400 nm, λem=505 nm) was measured every minute for thirty minutes at 37° C. The slope of the plot (time vs FLU) was taken. Relative inhibition was calculated as follows:

$$\% Relative Inhibition = ( \frac{{Slope}_{Enzyme Control} - {Slope}_{Sample}}{{Slope}_{Enzyme Control}} ) \times 100$$

The results are summarized in FIGS. 1 and 2, and Tables 1 and 2 below.

TABLE 1
Glycolic Acid

| | Average Inhibition | Standard |
|---|---|---|
| | (%) | Deviation |
| Positive Control (SPCK) | 100.0109 | 7.137924 |
| Negative Control (PBS) | 0 | 1.995331 |
| 0.125 wt % Glycolic Acid | 15.8949 | 1.471364 |
| 0.625 wt % Glycolic Acid | 23.894386 | 1.4483291 |
| 0.0125 wt % ATV | 12.48963 | 2.027787 |
| 0.025 wt % ATV | 14.96986 | 2.009879 |
| 0.0625 wt % ATV | 18.35813 | 2.03135 |
| 0.0125 wt % ATV + | 29.23064 | 2.068951 |
| 0.125 wt % Glycolic Acid | | |
| 0.025 wt % ATV + | 52.44359 | 2.078497 |
| 0.125 wt % Glycolic Acid | | |
| 0.0625 wt % ATV + | 64.94714 | 2.163293 |
| 0.125 wt % Glycolic Acid | | |
| 0.0125 wt % ATV + | 29.1853 | 2.074465 |
| 0.625 wt % Glycolic Acid | | |
| 0.025 wt % ATV + | 51.42889 | 2.079239 |
| 0.625 wt % Glycolic Acid | | |
| 0.0625 wt % ATV + | 59.62986 | 2.11721 |
| 0.625 wt % Glycolic Acid | | |

TABLE 2
Lactic Acid

| | Average Inhibition | Standard |
|---|---|---|
| | (%) | Deviation |
| Positive Control (SPCK) | 100.0808112 | 0.853338449 |
| Negative Control (PBS) | 0 | 0.950033704 |
| 0.125 wt % Lactic Acid | 8.507005217 | 0.741461688 |
| 0.625 wt % Lactic Acid | 24.09473075 | 0.844732585 |
| 0.0125 wt % ATV | 4.929862841 | 0.985914248 |
| 0.025 wt % ATV | 9.310938342 | 0.963233445 |
| 0.0625 wt % ATV | 11.95944762 | 0.96476709 |
| 0.0125 wt % ATV + | 14.81134343 | 1.122736831 |
| 0.125 wt % Lactic Acid | | |
| 0.025 wt % ATV + | 34.31431883 | 1.09021311 |
| 0.125 wt % Lactic Acid | | |
| 0.0625 wt % ATV + | 47.25971545 | 1.077350254 |
| 0.125 wt % Lactic Acid | | |
| 0.0125 wt % ATV + | 7.208154235 | 1.136390847 |
| 0.625 wt % Lactic Acid | | |
| 0.025 wt % ATV + | 29.76463436 | 1.08205185 |
| 0.625 wt % Lactic Acid | | |
| 0.0625 wt % ATV + | 46.44821182 | 1.091261707 |
| 0.625 wt % Lactic Acid | | |
| Positive Control (SPCK) | 100.0808112 | 0.853338449 |

Acetyl trifluoromethylphenyl valylglycine in combination with either glycolic acid or lactic acid was found to inhibit elastase hydrolysis of elastin at all tested concentrations in a dose-dependent manner, suggesting that acetyl trifluoromethylphenyl valylglycine can be combined with AHAs such as lactic acid and/or glycolic acid to synergistically reduce the breakdown of elastin in skin, and further improve skin elasticity.

### Example 2

### Effect on Skin Turnover

The effect of acetyl trifluoromethylphenyl valylglycine and/or chemical peels on skin turnover/desquamation, filaggrin expression, desmocollin-1 expression, kallikrein-related peptidase 5 expression, and terminal deoxynucleotidyl transferase dUTP nick end labeling was evaluated in an ex vivo skin model.

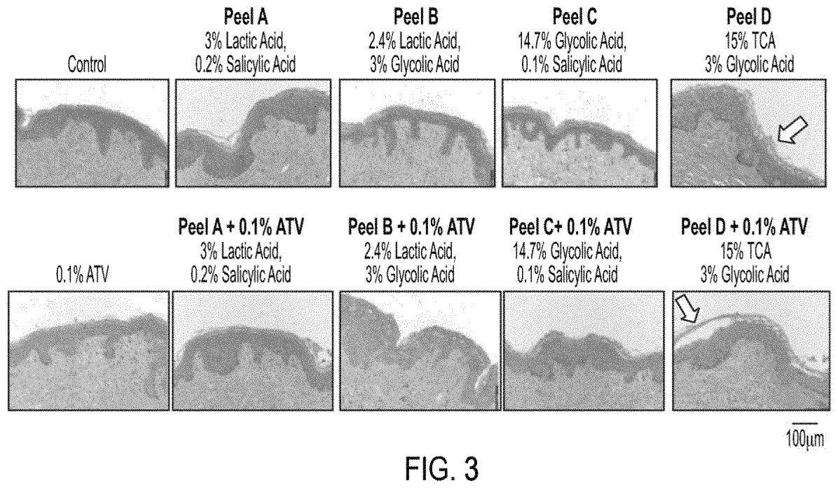

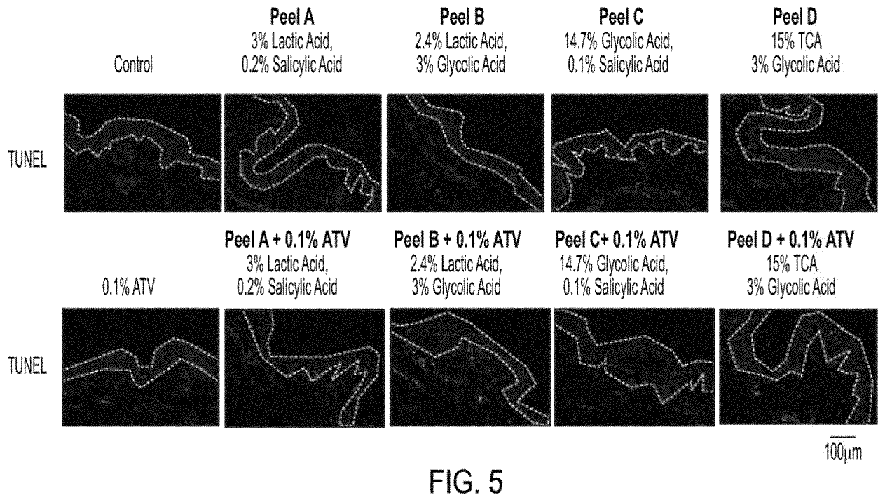

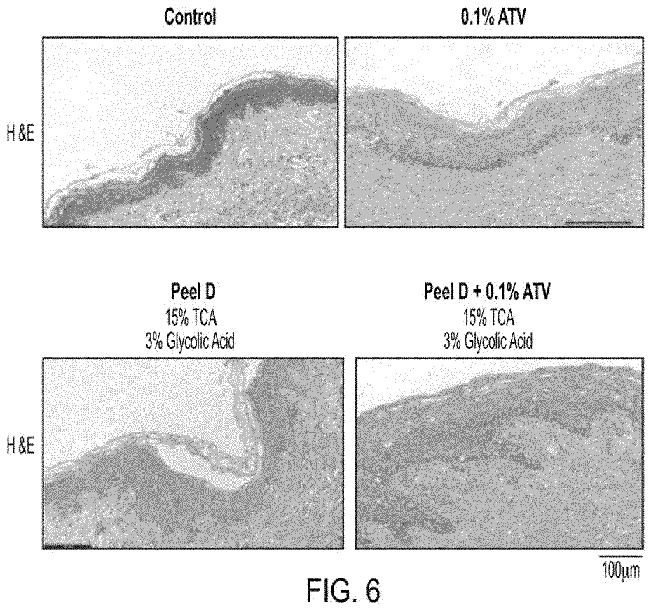

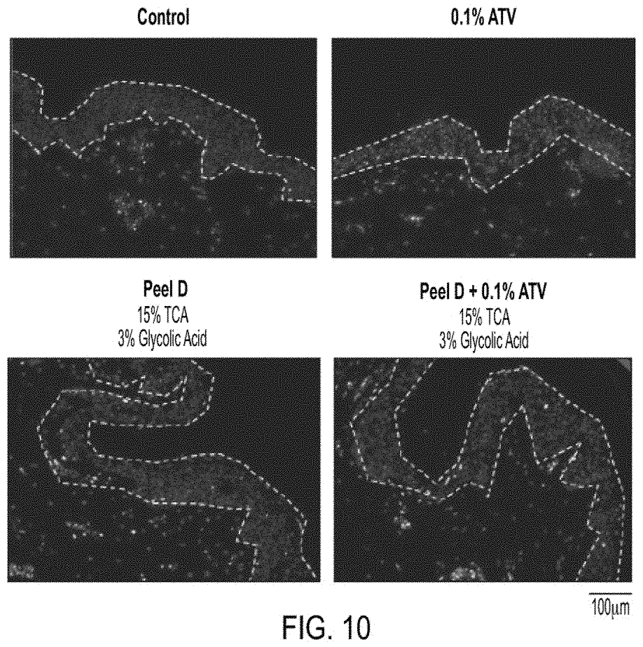

Fresh post-abdominoplasty normal human skin samples (3 lots, age 36-59 years old, Hispanic/Caucasian/African American, female) were acquired from BioIVT Inc. (Westbury, N.Y.). Tissue was defatted and cleaned of blood residue. Explant samples were then created using a 12 mm biopsy punch. Explants were subjected to two separate treatments of 5 uL of chemical peel formulations (Peels A-D, FIGS. 3-10), which were applied to the stratum corneum with a sterile cotton tip applicator for 10 minutes on Day 1 and Day 4 of culture. Skin biopsies not receiving treatment served as controls. Peel A in the figures contained 3 wt % lactic acid and 0.2 wt % salicylic acid. Peel B in the figures contained 3 wt % glycolic acid and 2.4 wt % lactic acid. Peel C in the figures is a commercially-available formulation having 14.7 wt % glycolic acid and 0.1 wt % salicylic acid (Peel C—Ingredients on label: Water, Glycolic Acid, Dicaprylyl Carbonate, Dimethicone, Sodium Hydroxide, Butylene Glycol, Propanediol, Glycerin, Phytic Acid, Cetyl PEG/PPG-10/1 Dimethicone, Aluminum Starch Octenylsuccinate, Nylon-12, Poly-glyceryl-4 Isostearate, Magnesium Sulfate, Jojoba Esters, Phenoxyethanol, Helianthus Annuus Seed Cera/Sunflower Seed Wax, Caprylyl Glycol, Disodium EDTA, Salicylic Acid, Acacia Decurrens Flower Cera/Accia Decurrens Flower Wax, Polyglycerin-3, Tocopherol, Pentaerythrityl tetra-di-t-butyl Hydroxyhydrocinnamate). Peel D in the figures is a commercially-available formulation having 15 wt % trichloroacetic acid and 3 wt % glycolic acid (Peel D—Ingredients on label: Water, Trichloroacetic Acid, Glycolic Acid, Sodium Lauryl Sulfate, Citric Acid).

After this time, samples were cleaned with sterile phosphate buffered saline (“PBS”). Skin explants were maintained at air-liquid interface at 37° C. and 5 wt %CO2 in two different treatment groups (n=3 per media type): 1) Dulbecco's Modified Eagle's Medium (DMEM) with 10 wt % Fetal Bovine Serum and 1 wt % Penicillin-Streptomycin, and 2 wt % ethanol serving as the vehicle control; and 2) Dulbecco's Modified Eagle's Medium with 10 wt % Fetal Bovine Serum, 1 wt % Penicillin-Streptomycin, and 0.1 wt % ATV in 2 wt % ethanol serving as the treatment group. Fresh media was replenished every other day except for weekends. Following a 7-day culture period, samples of all explants were taken for histological and immunohistochemical analysis. The remainder of the biopsy punches were used for corneocyte maturation analysis.

Skin explants were processed for haematoxylin and eosin, TUNEL and immunohistochemical staining (Reveal Biosciences, CA). H&E and TUNEL staining was performed using the standard protocol (Reveal Biosciences, CA).

Immunohistochemical staining was performed using a Leica Bond automated immunostainer (Reveal Biosciences, CA). Heat induced antigen retrieval was performed and non-specific antibody binding was blocked. Sections were incubated with the primary antibodies Mouse Monoclonal to Anti-Filaggrin (MA5-13441, Thermofisher, MA), Rabbit Polyclonal to Anti-Kallikrein 5 (LS-B15516-50, LS Bio, WA), or Rabbit Polyclonal to Anti-Desmocollin 1 (HPA075379, Sigma, MO). The primary staining was followed by rabbit anti-mouse IgG or anti-rabbit Poly-HRP-IgG respectively and were detected using the Bond Polymer Refine Detection kit (DS9800, Leica Biosystems, UK). All sections were then counterstained with a hematoxylin nuclear stain. Tissue samples were then embedded as formalin-fixed paraffin embedded (FFPE) blocks and imaged with an inverted microscope (Leica DM500, Germany).

FIGS. 3-10 disclose the images of the stained explants on the seventh day of culture following application of different chemical peels with or without acetyl trifluoromethylphenyl valylglycine. Tissue structure and integrity following chemical peel treatments was observed amongst the hematoxylin and eosin stained and immunohistochemical stained tissue of FIGS. 3-10. Tissues treated with 2 wt % ethanol in media in the figures serve as negative controls which demonstrate what untreated 2D ex-vivo histological cross-sections with no significant change to tissue health or evaluated biomarkers would look like. Peel D is a commercially-available professional grade peel that is included in FIGS. 3-5 for comparison with the at home use strength peels (Peels A-C). The images are representative of the turnover and renewal process of the skin after treatment with the corresponding chemical peels and/or ATV.

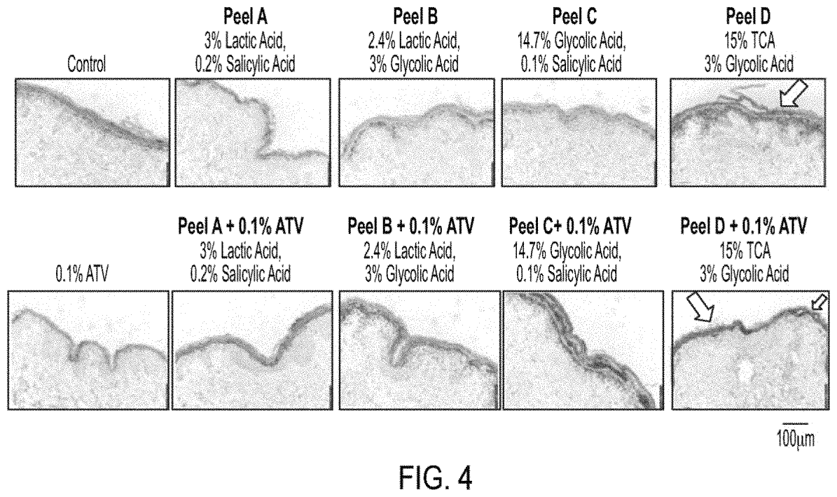

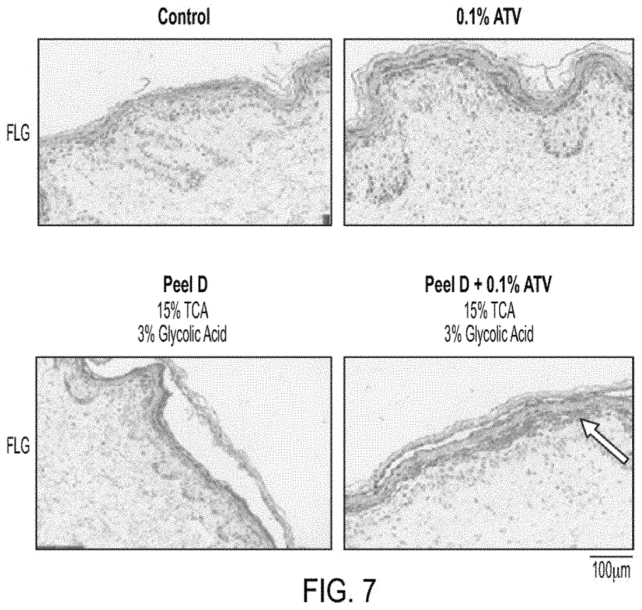

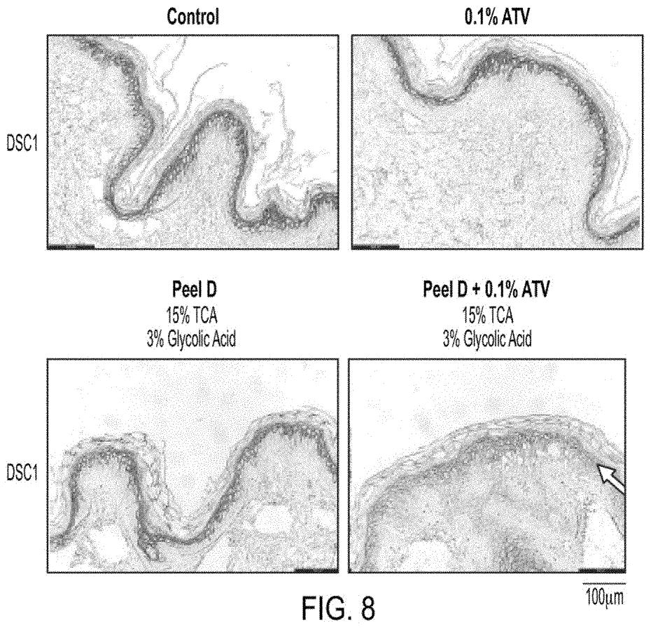

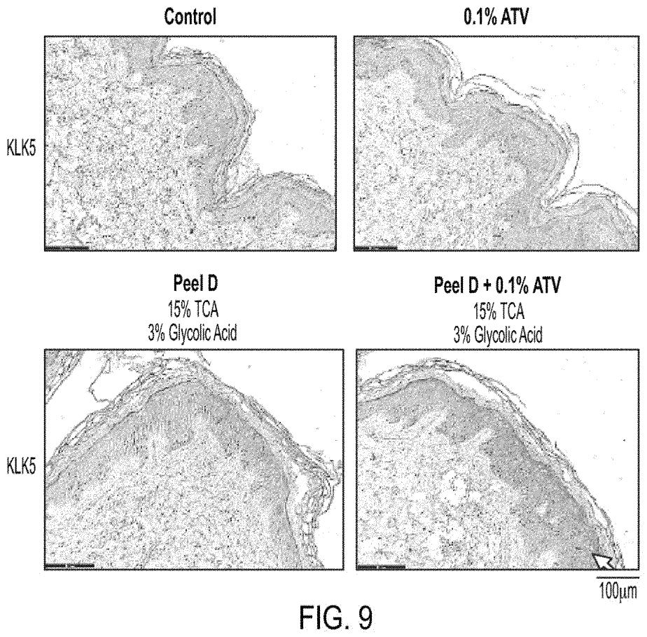

Several observations were made:
Treatment with at home-use chemical peels+acetyl trifluoromethylphenyl valylglycine (Peels B-C in FIG. 3) resulted in altered stratum corneum appearance as compared to vehicle control tissues and tissues that were not treated with acetyl trifluoromethylphenyl valylglycine in the H&E stained tissues, indicating an increase in epidermal renewal over treatments that do not include ATV. Peel C additionally demonstrated some signs of vacuole formation in upper epidermis. Treatment with a commercially-available professional chemical peel+acetyl trifluoromethylphenyl valylglycine (Peel D in FIG. 4) resulted in altered stratum corneum cohesion as compared to vehicle control tissues and tissues that were not treated with acetyl trifluoromethylphenyl valylglycine in the H&E stained tissues, indicating an increase in epidermal renewal after ATV+the professional peel treatment.
Treatment with chemical peels+acetyl trifluoromethylphenyl valylglycine (Peels B-C in FIG. 4, and D in FIG. 7) resulted in an increase in FLG expression in FLG-stained tissues as compared to the vehicle control sample tissue and tissues that were not treated with acetyl trifluoromethylphenyl valylglycine, indicating an increase in epidermal renewal over peel treatments that do not include ATV.
Treatment with a commercially-available professional chemical peel+acetyl trifluoromethylphenyl valylglycine (Peel D in FIG. 8) resulted in reduced DSC1 expression, which can help facilitate cell turnover, as compared to vehicle control tissues and tissues that were not treated with acetyl trifluoromethylphenyl valylglycine, indicating an increase in skin cell turnover after ATV+the professional peel treatment.
Treatment with a commercially-available professional chemical peel+acetyl trifluoromethylphenyl valylglycine (Peel D in FIG. 9) resulted in increased KLK5 expression (an enzyme that regulates desquamation) and increased signs of exfoliation as compared to vehicle control tissues and tissues that were not treated with acetyl trifluoromethylphenyl valylglycine, indicating an increase in desquamation after ATV+the professional peel treatment.
None of the chemical peel treated and/or acetyl trifluoromethylphenyl valylglycine treated tissues showed signs of increased TUNEL expression (FIGS. 3-10) as compared to vehicle control treated tissues, which suggests that the treated skin cells are not undergoing increased rates of DNA degradation as would otherwise occur during the late stages of apoptosis.

Thus, the ex vivo skin model results demonstrate that acetyl trifluoromethylphenyl valylglycine can be paired synergistically with chemical peels having various components and amounts thereof to increase or improve skin renewal.

### Example 3

### Penetration into Skin

Human skin samples were obtained from abdominal surgery. Excess subcutaneous fat was removed, if necessary. Skin samples were excised and cut into pieces of around 2 cm×2 cm. Thickness was measured and transepidermal water loss (TEWL) was measured. The human skin was included in the study if the TEWL was between 0.5 and 13 g/m²/h for abdominal skin. The 2 cm² skin samples were kept in a static cell in a diffusion chamber in a Franz cell horizontal cell clamp system (Crown Glass Company, Inc.) and maintained at a constant temperature of 32° C.±1° C. The static cells consist of a donor chamber and a receptor chamber with a 7 mL receptor fluid compartment separated by the skin.

The cells were placed on a magnetic stirrer which was integrated with a fixed temperature water bath maintained in order to have 32° C.±1° C. at the skin surface. The receptor compartment of each cell was filled with the receptor fluid (PBS) and was continuously stirred. The skin samples were placed on the receptor compartment. The donor compartment was then placed onto the skin samples. After confirming the absence of air-bubbles, a clamp was placed to link both compartments. Agitation of the receptor fluid was started.

A composition containing 2 wt % acetyl trifluoromethylphenyl valylglycine was applied to the skin sample at a rate of 10 mg/cell using a positive displacement pipette and the exact amount applied was determined by weight then recorded (Sartorius Entris II Essential Analytical balance, Thermo-Fisher).

The experiment started immediately after the application of the preparation on the skin surface and ended 16 hours after application. The preparation was washed off the skin samples 16 hours after application.

The skin was then stripped with consistent weight and time of application. A maximum of 20 strips was performed on each skin sample and the strips were pooled by 5. The strips samples were stored with solvent (methanol) at −20° C. for a maximum of 5 days before being analyzed. Using the scalpel blade, the epidermis (E) and dermis (D) corresponding to the application area were cut and then separated by heating. Epidermis (E) and dermis (D) were weighed. The remaining skin (RS) was cut in four parts. The RS vials were stored with solvent (methanol) at −20° C. for a maximum of 7 days before being analyzed. Dermis and epidermis samples were stored with solvent at −20° C. for a maximum of 7 days before being analyzed. The receptor fluid was taken 16 hours after the application and the samples were analyzed.

Receptor fluid samples were stored at −20° C. for a maximum of 6 days before being analyzed. The donor compartment, the upper seal and the scalpel blade (LM) were placed in a flask with methanol and shaken for 2 hours. Samples were stored at −20° C. for a maximum of 5 days before being analyzed.

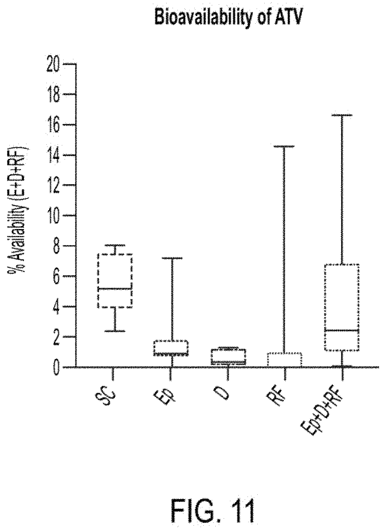

The final extract was analyzed by LC-MS/MS. Results are provided in FIG. 11. The skin penetration level (EP+D+RF in FIG. 11) of acetyl trifluoromethylphenyl valylglycine was calculated to be about 4 wt % of the 2 wt % acetyl trifluoromethylphenyl valylglycine preparation. Thus, about 0.08 wt % acetyl trifluoromethylphenyl valylglycine penetrated through the stratum corneum. As such, the dermal dose of the 2 wt % acetyl trifluoromethylphenyl valylglycine preparation based on in vitro functionality and penetration level was calculated to be at least about 0.08 wt %.

Thus, the application of the 2% acetyl trifluoromethylphenyl valylglycine composition to skin resulted in about 0.8% penetrating past the stratum corneum barrier and into the fractions of epidermis, dermis, and receptor fluid. The level of penetrated acetyl trifluoromethylphenyl valylglycine correlates to the approximate level of the dosed acetyl trifluoromethylphenyl valylglycine in the elastase (Example 1) and ex vivo skin (Example 2) experiments above.

### Example 4

### Clinical Studies

A monocentric, full-face, randomized, and double-blinded study was conducted to evaluate the clinical efficacy and consumer perception of three home-care regimens on improving facial dyschromia and aging signs in Caucasian healthy women. Eighty-six (86) overall healthy Caucasian women, aged 40-65 years (mean age 57.85 years), presenting with mild to moderate sign of aging with Fitzpatrick skin phototype II-III were included in this study. Pregnant or lactating women who have used retinol or related product and/or received treatment for chemical peel, laser, photo facials, dermabrasion, botulinum toxin, injectable fillers, or other skin tightening treatments within 6 months of the study start date were excluded. Once enrolled, subjects were asked to stop their regular skin care regime. The subjects in Table 1 were trained on self-application of all test products prior to study start.

TABLE 3
Demographics of Enrolled Subjects

| Group 1: Retinol product | Group 2: ATV and Home Peel |
|---|---|
| N = 46 | N = 40 |
| Age mean (years): 58.6 | 19 Fitz II and 21 Fitz III |
| Retinol Naïve users: | Retinol Naïve users: |
| 38; Retinol users: 8 | 33; Retinol users: 7 |

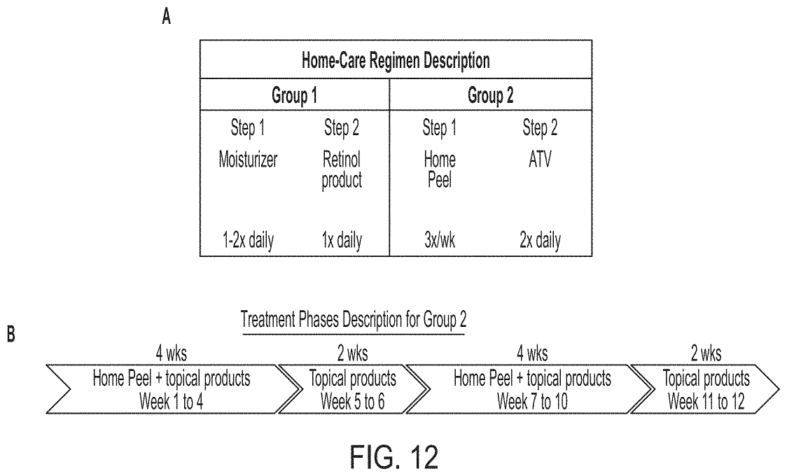

The study consisted of a 7-day washout period, followed by 12 weeks of treatment. Subjects were provided with auxiliary products (cleanser, moisturizer, and sunscreen with SPF 30) to use during the washout period. Cleanser and sunscreen were also used during the 12 weeks treatment. Following the washout period, subjects were randomized into two home-care regimen groups, as illustrated in FIG. 12(A). The regimens are described below:

Regimen 1 consisted of a moisturizer, applied every morning; a retinol-containing product, applied every third night during the first week post-washout period, followed by every two days during the second week, then applied nightly for the remained of the study duration.

Regimen 2 consisted of a 2% acetyl trifluoromethylphenyl valylglycine composition, applied daily morning and evening, and a 0.45% salicylic acid/11.11% lactic acid chemical peel applied three times a week in the evening.

Regimen Group 1 used the same product routine throughout the 12 weeks of treatment. For Regimen Group 2, the 12-week treatment periods were divided into two phases, completed twice (FIG. 12(B)): 4 weeks of using the home chemical peel in combination with all of the topical products (Week 1 to Week 4 and Week 7 to Week 10), followed by 2 weeks of using the topical products, but not the chemical peel treatment (Week 5 to Week 6 and Week 11 to Week 12).

The subjects were evaluated for clinical efficacy and tolerability at baseline and weeks 2, 4, 6, 10, and 12. Photoaging parameters were assessed globally using a modified Griffith's 10-point scale according to the following numeric definition (half-point scores were used as necessary for accuracy): 0=none, 1-3=mild, 4-6=moderate, and 7-9=severe. Crow's feet and nasolabial fold wrinkles were assessed globally using the Caucasian Skin Aging Atlas volume 1 scales, ranging from 0-5 (score of 0.4 and 0.6 were used as necessary for accuracy). Tolerability was objectively assessed at each visit by investigator by evaluating signs of erythema, dryness, scaling, and peeling. In addition, subjects self-report of the degree of burning, stinging, itching, dryness, tightness, or tingling of facial skin are also recorded. Both clinical grading and subjective tolerability assessments were measured using a 3-point scale (half-point scores were used as necessary for accuracy): where 0=none; 1=mild; 2=moderate; and 3=severe.

Self-assessment questionnaires were given to subjects to evaluate their perception on facial skin condition, product efficacy and sensory at baseline, weeks 4, 6 and 12. according to the following system: agree completely, agree somewhat, neither agree nor disagree, disagree somewhat, and disagree completely.

For clinical assessment of efficacy and tolerance parameters, a frequentist and bayesian linear mixed model were used to analyze the mean difference in change from baseline with baseline, treatment, time and treatment-time interaction as fixed effects and subject as random effect to obtain differences in treatment effects and time effect. Effect size and p-values were both used to interpret significant differences (p-values<0.05 were considered statistically significant and classes of effect size).

Tolerability Results:

Table 2 below discloses mean Scores on 0-3 scale (0=none, 1=mild, 2=moderate, 3=severe).

TABLE 4
Skin Tolerance Grading (mean score) across
Home-Care Regimen Groups

| | | Week 6 | | Week 12 | |
|---|---|---|---|---|---|
| | | Group 1 | Group 2 | Group 1 | Group 2 |
| | | (n = 32 | (n = 15 | (n = 16 | (n = 9 |
| | Parameters | reactions) | reactions) | reactions) | reactions) |
| Objective | Eythema | 1 (n = 25) | 1 (n = 12) | 1 (n = 15) | 1.2 |
| Assessment | Peeling | 1.3 (n = 4) | 1 (n = 1) | 1 (n = 1) | 0 |
| (Investigator) | Dryness | 1.5 (n = 2) | 1 (n = 1) | 0 | 0 |
| | Edema | 1.5 (n = 1) | 0 | 0 | 0 |
| Subjective | Stinging/ | 1 (n = 1) | 0 | 0 | 0 |
| Assessment | Burning/ | | | | |
| | Itching | | | | |
| | Tingling | 0 | 0 | 0 | 0 |
| (Subject | Tightness | 0 | 0 | 0 | 0 |
| Reported) | | | | | |

More subjects in Group 1 as compared to Group 2 experienced mild to moderate local intolerances at Week 6, with erythema signs as the driving factor. One subject also self-reported stinging, burning, and itching. By Week 12, only mild erythema reactions were clinically observed. Approximately half as many subjects in Group 2 experienced mild to moderate local intolerances at Week 12, as compared to Group 1.

Adverse Events:

Overall, 8 subjects experienced a total of 8 non-serious adverse events (AEs) that were considered un-related to product regimen, and all resolved without sequelae. These subjects were withdrawn from the study by the investigator.

Efficacy Evaluations:

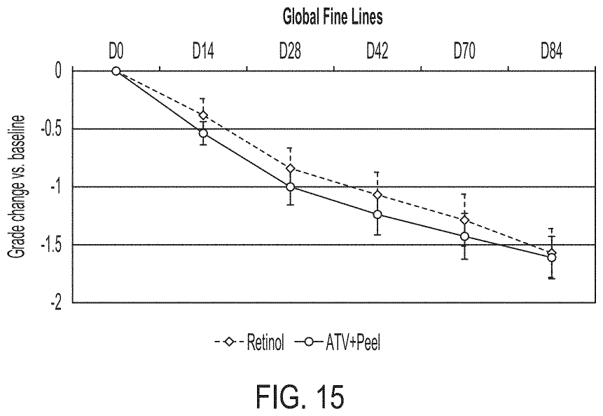

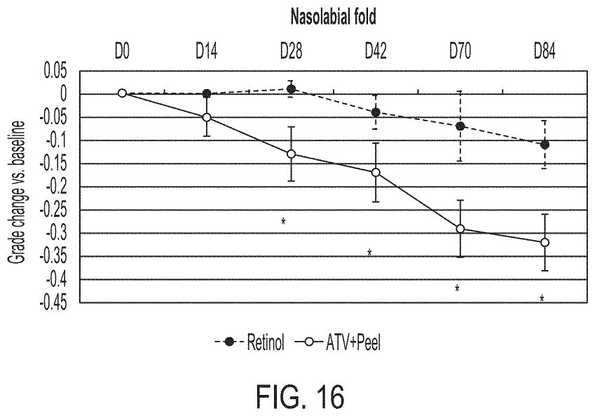

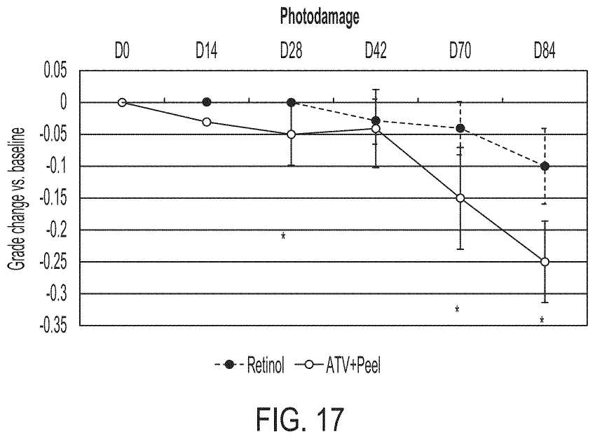

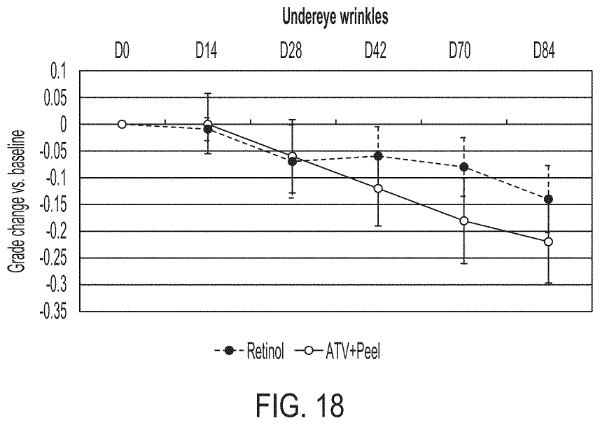

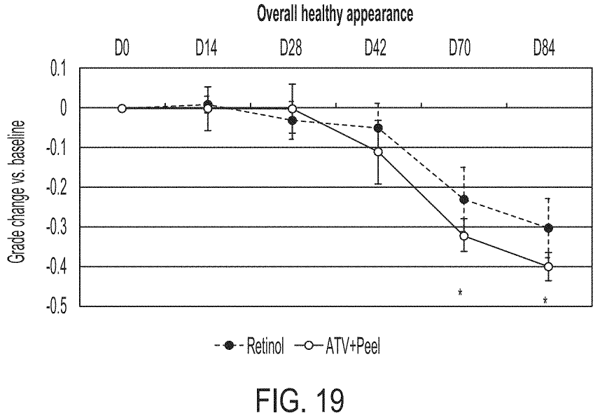

Clinical evaluation of anti-aging parameters demonstrated significant improvement over baseline for both skincare regimen groups at Week 2 and 4 for global fine lines (FIG. 15). By Week 6, Group 2, which included the chemical peel, showed a significant improvement in photodamage and nasolabial fold wrinkles appearance from baseline (FIGS. 16-17). By Week 12, all treatment groups demonstrated a significant improvement in all parameters assessed over baseline, as illustrated in FIGS. 15-19.

Treatment comparisons in the figures demonstrated that Group 2 (chemical peel and acetyl trifluoromethylphenyl valylglycine), was significantly better at improving nasolabial folds than Group 1 (retinol-containing product) by Week 4, and photodamage by Week 7. Group 2 significantly outperformed Group 1 by Week 12 in all assessed anti-aging parameters (FIGS. 15-19).

The anti-aging routine containing acetyl trifluoromethylphenyl valylglycine+at a chemical peel treatment delivers superior anti-aging and skin quality benefits in an accelerated kinetic manner, as compared to retinol use alone.

1-177. (canceled)

178. A method for treating skin, the method comprising applying to the skin:
(a) acetyl trifluoromethylphenyl valylglycine; and
(b) at least one peeling agent,
wherein the (a) acetyl trifluoromethylphenyl valylglycine is applied to the skin simultaneously with the (b) at least one peeling agent, or consecutively within about 48 hours before and/or after applying the (b) at least one peeling agent to the skin.

179. The method of claim 178, wherein the peeling agent is applied to the skin at least one time per week.

180. The method of claim 178, wherein the peeling agent is applied to the skin at least two times per week.

181. The method of claim 178, wherein the (a) acetyl trifluoromethylphenyl valylglycine is applied to the skin within about 24 hours before and/or after applying the (b) at least one peeling agent to the skin.

182. The method of claim 178, wherein the method comprises applying to the skin a composition comprising acetyl trifluoromethylphenyl valylglycine in an amount ranging from about 0.001 to about 25 wt % relative to the total weight of the composition in which it is present.

183. The method of claim 178, wherein the at least one peeling agent is a chemical peeling agent.

184. The method of claim 183, wherein:
the at least one chemical peeling agent is applied to the skin at least two times per week, and
the at least one chemical peeling agent is applied to the skin at least one time per day on non-consecutive days.

185. The method of claim 184, wherein the chemical peeling agent is applied to the skin at least three times per week for about four weeks, followed by a rest period of about two weeks wherein the chemical peeling agent is not applied to the skin.

186. The method of claim 183, wherein the at least one chemical peeling agent comprises at least one compound chosen from α-hydroxy acids, β-hydroxy acids, trichloroacetic acid, or mixtures thereof.

187. The method of claim 178, wherein the at least one peeling agent is a composition comprising at least one chemical peeling agent chosen from α-hydroxy acids, wherein the total amount of α-hydroxy acids in the composition ranges from about 0.1 to about 25 wt %, relative to the total weight of the composition in which it is present.

188. The method of claim 178, wherein the at least one peeling agent is a composition comprising from about 0.1 to about 25 wt % lactic acid, relative to the total weight of the composition in which it is present.

189. The method of claim 178, wherein the at least one peeling agent is a composition comprising from about 0.1 to about 25 wt % glycolic acid, relative to the total weight of the composition in which it is present.

190. The method of claim 178, wherein the at least one peeling agent is a composition comprising at least one β-hydroxy acid in an amount ranging from about 0.01 to about 2 wt %, relative to the total weight of the composition in which it is present.

191. The method of claim 178, wherein the at least one peeling agent is a composition comprising salicylic acid or a derivative thereof in an amount ranging from about 0.01 to about 2 wt %, relative to the total weight of the composition in which it is present.

192. The method of claim 178, wherein the at least one peeling agent is a composition comprising trichloroacetic acid in an amount ranging from about 1 to about 25 wt %, relative to the total weight of the composition in which it is present.

193. The method of claim 178, comprising applying to the skin:
(a) acetyl trifluoromethylphenyl valylglycine at least one time per day; and
(b) at least one chemical peeling agent at least one time every two to three days.

194. The method of claim 193, wherein the method is repeated for a period of time of at least about two weeks.

195. The method of claim 193, wherein the method is repeated for a period of time of at least about four weeks, followed by a rest period of at least about two weeks wherein the chemical peeling agent is not applied to the skin.

196. A composition comprising:
(i) acetyl trifluoromethylphenyl valylglycine; and
(ii) at least one chemical peeling agent,
wherein the (a) acetyl trifluoromethylphenyl valylglycine, and the (b) at least one chemical peeling agent are present in the composition in amounts effective to stimulate skin renewal of the skin.

197. A kit comprising:
(i) a first chamber or container comprising acetyl trifluoromethylphenyl valylglycine; and
(ii) a second chamber or container comprising at least one chemical peeling agent.
